(19) United States
(12) Patent Application Publication
(10) Pub. No.: US 2022/0341923 A1
(43) Pub. Date: Oct. 27, 2022

(54) MULTI-PLEX ASSAY PLATES AND METHODS OF MAKING

(71) Applicant: Meso Scale Technologies, LLC., Rockville, MD (US)

(72) Inventors: John H. KENTEN, Boyds, MD (US); Galina NIKOLENKO, Damascus, MD (US)

(21) Appl. No.: 17/728,292

(22) Filed: Apr. 25, 2022

Related U.S. Application Data
(60) Provisional application No. 63/179,731, filed on Apr. 26, 2021.

(51) Int. Cl. G01N 33/543; G01N 21/76; G01N 33/53; G01N 33/569; G01N 33/58

(52) U.S. Cl. CPC G01N 33/54306; G01N 21/76; G01N 33/5308; G01N 33/56983; G01N 33/582; G01N 2021/7786

(57) ABSTRACT

Described herein is a method of preparing a bifunctional assay surface. In particular, a method is provided for preparing an assay surface that includes a primary reagent and a secondary reagent. In one aspect, the primary and secondary reagents are immobilized on the assay surface by different surface chemistries. In one aspect, a method is provided for preparing an assay surface that includes a proteinaceous primary reagent and a thiol-containing secondary reagent. In one aspect, a method is provided for preparing an assay surface that includes a capture-target hybrid and a thiol-containing secondary reagent.

### FIELD OF THE INVENTION

Described herein are methods for preparing a bifunctional assay surface.

### BACKGROUND OF THE INVENTION

Numerous methods and systems have been developed for conducting chemical, biochemical and/or biological assays in which the presence and/or quantity of one or more analytes of interest in a sample is determined by detecting the participation of the analyte(s) directly or indirectly in a binding reaction, for example, an antigen-antibody reaction, a nucleic acid hybridization or a receptor-ligand reaction. In some approaches, participation of the analyte in the binding reaction is indicated by measuring an observable label attached to one or more binding materials. These methods and systems are used in a variety of applications, including, but not limited to, medical diagnostics, food and beverage testing, environmental monitoring, manufacturing quality control, drug discovery and basic scientific research.

Assays to measure the level of a single analyte (singleplex assays) have been a mainstay of biological research for decades. Assays can also be performed to measure multiple analytes in parallel (multiplex assays) to reduce workflow and sample volume requirements. There are two formats typically used for multiplex assays: immobilization on a solid surface that includes one or more binding domains; and immobilization on beads or particles in which the binding domain for each analyte is on a different bead or particle.

For some assays, it may be desirable to have a bifunctional assay surface in which one or more binding domains include one or more different reagents. However, there remains a need for methods for making bifunctional assay surfaces for analysis of one or more target analytes in a sample.

### SUMMARY OF THE INVENTION

Provided herein are methods for preparing a bifunctional assay surface. In one aspect, the method includes a direct coating method in which a coating solution that includes a primary reagent and a secondary reagent is dispensed onto an assay surface to form a coated assay surface. In one aspect, the method includes an overcoating method in which an overcoating solution that includes a secondary reagent is dispensed onto an assay surface on which a primary reagent is immobilized to form a coated assay surface. In one aspect, the primary reagent and the secondary reagent are immobilized on the assay surface through different surface chemistries.

In one aspect, a direct method for preparing a bifunctional assay surface is provided in which a coating solution that includes a primary reagent and a secondary reagent is dispensed onto an assay surface to form a coated assay surface. In one aspect, the primary reagent is a proteinaceous primary reagent. In one aspect, the primary reagent is a capture-target hybrid. In one aspect, the secondary reagent is a thiol-containing secondary reagent. In one aspect, the secondary reagent is a thiolated oligonucleotide. In one aspect, the assay surface is a carbon-containing assay surface. In one aspect, the method includes incubating the coated assay surface under conditions in which the primary reagent and secondary reagent are immobilized on the carbon-containing assay surface. In one aspect, the thiol-containing secondary reagent is immobilized on the carbon-containing assay surface through a thiol group. In one aspect, the thiol-containing secondary reagent is immobilized on the carbon-containing assay surface through the formation of a covalent bond between the thiol group and a reactive functional group on the assay surface. In one aspect, the method includes dispensing from about 10 nl to about 100 nl, about 25 nl to about 75 nl, about 40 nl to about 60 nl, or about 50 nl of the coating solution onto the assay surface.

In one aspect, the coating solution includes about 100 μg/ml to 750 μg/ml, about 500 μg/ml to 750 μg/ml, or about 100 μg/ml to about 400 μg/ml, proteinaceous primary reagent or capture-target hybrid. In one aspect, the coating solution includes from about 15 nM to about 1500 nM thiol-containing secondary reagent. In one aspect, the coating solution includes from about 1.5 nM to about 1500 nM thiol-containing secondary reagent. In one aspect, the coating solution includes from about 1.5 nM to about 10 nM thiol-containing secondary reagent. In one aspect, the coating solution includes from about 1.5 nM to about 15 nM thiol-containing secondary reagent. In one aspect, the coating solution includes from about 15 nM to about 30 nM thiol-containing secondary reagent. In one aspect, the coating solution includes from about 30 nM to about 50 nM thiol-containing secondary reagent. In one aspect, the coating solution includes from about 50 nM to about 100 nM thiol-containing secondary reagent. In one aspect, the coating solution includes from about 100 nM to about 150 nM thiol-containing secondary reagent. In one aspect, the coating solution includes from about 150 nM to about 300 nM thiol-containing secondary reagent. In one aspect, the coating solution includes from about 300 nM to about 500 nM thiol-containing secondary reagent. In one aspect, the coating solution includes from about 500 nM to about 750 nM thiol-containing secondary reagent. In one aspect, the coating solution includes from about 750 nM to about 1000 nM thiol-containing secondary reagent. In one aspect, the coating solution includes from about 1000 nM to about 1500 nM thiol-containing secondary reagent.

In one aspect, the coating solution includes from about 100 μg/ml to about 500 μg/ml, 500 μg/ml to about 750 μg/ml, or 750 μg/ml to about 1000 μg/ml streptavidin; from about 15 nM to about 1500 nM thiol-containing secondary reagent; from about 0.01% to about 0.1% TRITON X-100; optionally from about 0.1% to about 0.5% trehalose in phosphate buffered saline; optionally about 400 μg/ml to about 750 μg/ml of an additional proteinaceous component, including but not limited to Bovine Serum Albumin (BSA), Human Serum Albumin (HSA), Protein A, and Protein G and from about 1 mM EDTA to about 20 mM EDTA. In one aspect, the coating solution includes from about 100 μg/ml to about 400 μg/ml, 400 μg/ml to about 750 μg/ml, or 750 μg/ml to about 1000 μg/ml streptavidin; from about 15 nM to about 1500 nM thiol-containing secondary reagent; about 0.03% TRITON-X-100; optionally about 0.4% trehalose in phosphate buffered saline; optionally about 400 μg/ml to about 750 μg/ml of an additional proteinaceous component, including but not limited to BSA, HSA, Protein A, and Protein G, and about 10 mM EDTA. In one aspect, the coating solution includes from about 100 μg/ml to about 500 μg/ml, 500 μg/ml to about 750 μg/ml, or 750 μg/ml to about 1000 μg/ml streptavidin; from about 1500 nM to about 2500 nM thiol-containing secondary reagent; about 0.03% TRITON-X-100; optionally about 0.4% trehalose in phosphate buffered saline; optionally about 400 μg/ml to about 750 μg/ml of an additional proteinaceous component, including but not limited to BSA, HSA, Protein A, and Protein G, and about 10 mM EDTA. In one aspect, phosphate buffered saline includes Dulbecco's phosphate buffered saline (DPBS) that includes 2.67 mM KCl, 1.47 mM KH2PO4, 8.1 mM Na2HPO4, and 138 mM NaCl. In one aspect, the method includes drying the coated assay surface overnight at room temperature. In one aspect, the method includes drying the coated assay surface at a controlled humidity of about 40%.

In one aspect, the coating solution includes from about 100 μg/ml to about 400 μg/ml, 400 μg/ml to about 750 μg/ml, or 750 μg/ml to about 1000 μg/ml proteinaceous primary reagent or capture-target hybrid; from about 15 nM to about 1500 nM thiol-containing secondary reagent; from about 0.01% to about 0.1% TRITON X-100 and optionally from about 0.1% to about 0.5% trehalose in phosphate buffered saline; optionally about 400 μg/ml to about 750 μg/ml of an additional proteinaceous component, including but not limited to BSA, HSA, Protein A, and Protein G and from about 1 mM EDTA to about 20 mM EDTA. In one aspect, the coating solution includes from about 100 μg/ml to about 400 μg/ml, 400 μg/ml to about 750 μg/ml, or 750 μg/ml to about 1000 μg/ml proteinaceous primary reagent or capture-target hybrid; from about 15 nM to about 1500 nM thiol-containing secondary reagent; about 0.03% TRITON X-100 and about 0.4% trehalose in phosphate buffered saline; optionally about 400 μg/ml to about 750 μg/ml of an additional proteinaceous component, including but not limited to BSA, HSA, Protein A, and Protein G and about 10 mM EDTA. In one aspect, phosphate buffered saline includes Dulbecco's phosphate buffered saline (DPBS) that includes 2.67 mM KCl, 1.47 mM KH₂PO₄, 8.1 mM Na₂HPO₄, and 138 mM NaCl. In one aspect, the method includes drying the coated assay surface overnight at room temperature. In one aspect, the method includes drying the coated assay surface at a controlled humidity of about 40%.

In one aspect, an overcoating method for preparing a bifunctional assay surface is provided in which an overcoating solution that includes a secondary reagent is dispensed onto an assay surface on which one or more primary reagents are immobilized to form a coated assay surface. In one aspect, the assay surface is a carbon-containing assay surface. In one aspect, the primary reagent is a proteinaceous primary reagent. In one aspect, the primary reagent is a capture-target hybrid. In one aspect, the secondary reagent is a thiol-containing secondary reagent. In one aspect, the secondary reagent is a thiolated oligonucleotide. In one aspect, the method includes incubating the coated assay surface under conditions in which the thiol-containing secondary reagent is immobilized on the carbon-containing assay surface through a thiol group. In one aspect, the thiol-containing secondary reagent is immobilized on the carbon-containing assay surface through the formation of a covalent bond between the thiol group and a reactive functional group on the assay surface. In one aspect, the method includes dispensing from about 10 μL to about 100 μL, about 25 μL to about 75 μL, or about 30 μL to about 50 μL of the overcoating solution onto the assay surface. In one aspect, the overcoating solution includes from about 0.01 μM to about 20 μM thiol-containing secondary reagent. In one aspect, the overcoating solution includes a buffer selected from: Deprotection-Conjugation Buffer (10 mM phosphate, pH 7.4, 150 mM NaCl, 10 mM EDTA), 1×PBS (phosphate buffered saline)/10 mM EDTA (ethylenediaminetetraacetic acid) or Diluent 100 (Meso Scale Diagnostics, LLC). In one aspect, the method includes incubating the coated assay surface from about 1 hours to about 5 hours, about 1 hours to about 3 hours, or about 2 hours at room temperature while shaking at from about 500 rpm to about 725 rpm. In one aspect, the method includes incubating the coated assay surface at room temperature while shaking at about 705 rpm.

In one aspect, the assay surface is part of a multi-well plate. In one aspect, the carbon-containing assay surface is part of a multi-well plate.

In one aspect, one or more primary reagents are immobilized on the assay surface in discrete binding domains. In one aspect, one or more proteinaceous primary reagents or capture-target hybrids are immobilized on a carbon-containing assay surface in discrete binding domains. In one aspect, a plurality of proteinaceous primary reagents or capture-target hybrids are immobilized on a carbon-containing assay surface. In one aspect, the plurality of primary reagents include primary reagents that bind to different targets than each other. In one aspect, the primary reagents are immobilized on the assay surface in an array. In one aspect, one or more proteinaceous primary reagents or capture-target hybrids are immobilized on a carbon-containing assay surface in an array. In one aspect, one or more secondary reagents are immobilized on the assay surface. In one aspect, one or more thiol-containing secondary reagents are immobilized on the assay surface.

In one aspect, the primary reagent is immobilized in a first binding domain and the secondary reagent is immobilized in a second binding domain. In one aspect, a plurality of proteinaceous primary reagents or capture-target hybrids are immobilized in a plurality of primary binding domains and a plurality of thiol-containing secondary reagents are immobilized in a plurality of secondary binding domains. In one aspect, each primary binding domain includes a unique primary reagent. In one aspect, each secondary binding domain includes a unique secondary reagent. In one aspect, each secondary binding domain includes a common secondary reagent.

In one aspect, each binding domain includes a primary reagent and a secondary reagent. In one aspect, each binding domain includes a proteinaceous primary reagent and a thiol-containing secondary reagent. In one aspect, each binding domain includes a capture-target hybrid and a thiol-containing secondary reagent. In one aspect, each binding domain includes a unique primary reagent. In one aspect, each binding domain includes a unique secondary reagent. In one aspect, each binding domain includes a common secondary reagent. In one aspect, each binding domain includes a unique primary reagent and a unique secondary reagent. In one aspect, each binding domain includes a unique primary reagent and a common secondary reagent.

In one aspect, the primary reagent is non-covalently immobilized on the carbon-containing assay surface. In one aspect, the primary reagent is a proteinaceous primary reagent or capture-target hybrid that is non-covalently immobilized on a carbon-containing assay surface. In one aspect, the primary reagent is covalently immobilized on a carbon-containing assay surface. In one aspect, the primary reagent is a proteinaceous primary reagent or capture-target hybrid that is covalently immobilized on a carbon-containing assay surface. In one aspect, the primary reagent or capture-target hybrid is immobilized on the assay surface via a binding pair. In one aspect, the primary reagent is a proteinaceous primary reagent or capture-target hybrid that is immobilized on the carbon-containing assay surface via a binding pair. In one aspect, the primary reagent includes a first member of a binding pair and the assay surface includes a second member of the binding pair. In one aspect, the primary reagent includes biotin and the assay surface includes avidin or streptavidin. In one aspect, the primary reagent includes a proteinaceous capture reagent. In one aspect, the proteinaceous capture reagent includes an antibody, an antigen-binding fragment of an antibody or a receptor. In one aspect, the secondary reagent is a thiol-containing secondary reagent. In one aspect, the thiol-containing secondary reagent includes a thiolated oligonucleotide.

In one aspect, the assay surface is a carbon-containing assay surface that is treated to introduce one or more maleimide groups before the thiol-containing secondary reagent is dispensed onto the carbon-containing assay surface. In one aspect, the carbon-containing assay surface is treated with an amine-to-sulfhydryl crosslinker that includes SM(PEG)ₙ, wherein n=2 to 24 (ThermoFisher Scientific). In one aspect, n=4.

In one aspect, one or more secondary reagents are immobilized on the assay surface. In one aspect, a plurality of secondary reagents are immobilized on an assay surface. In one aspect, a plurality of thiol-containing secondary reagents are immobilized on a carbon-containing assay surface. In one aspect, the secondary reagent includes a thiolated oligonucleotide. In one aspect, the thiolated oligonucleotide has a length from about 1 to about 100, about 5 to about 50, about 5 to about 10 nucleotides, or about 10 to about 30 nucleotides. In one aspect, the thiolated oligonucleotide includes an oligonucleotide attached to a thiol group through a linker. In one aspect, the linker includes from about 3 to about 20 atoms or molecules or units. In one aspect, the thiolated oligonucleotide includes an oligonucleotide sequence having a 5′- and a 3′-end and a thiol group incorporated at the 5′ end (a 5′-terminal thiolated oligonucleotide), at the 3′ end (a 3′-terminal thiolated oligonucleotide), at an internal position of the oligonucleotide, or a combination thereof. In one aspect, the thiolated oligonucleotide includes deoxyribonucleic acid (DNA), ribonucleic acid (RNA), 2′-O-methylnucleotide (2′-OMe), 2′-O-methoxyethyl nucleotide (2′-MOE), a 2′-deoxy-2′-fluoro ribonucleotide (2′-F-RNA), a bridged nucleic acid (BNA), locked nucleic acid (LNA), peptide nucleic acid (PNA), or a combination thereof. In one aspect, the thiolated oligonucleotide includes one or more non-natural nucleotide bases. In one aspect, the non-natural nucleotide base is selected from: 2,6-Diaminopurine (2-Amino-dA); 5-Methyl deoxycytidine, Super T (5-hydroxybutynl-2′-deoxyuridine), or a combination thereof.

In one aspect, the secondary reagent is a thiol-containing secondary reagent that includes a thiolated member of a binding pair. In one aspect, the secondary reagent includes thiolated biotin (Thiol-Biotin). In one aspect, the secondary reagent includes thiolated polyethylene glycol (Thiol-PEG). In one aspect, the thiolated polyethylene glycol further includes biotin (Thiol-PEG-Biotin).

In one aspect, the secondary reagent is a thiol-containing secondary reagent that includes thiolated fluorescein isothiocyanate (FITC).

In one aspect, the primary reagent includes a capture reagent that specifically binds to or comprises a target molecule or target analyte. In one aspect, the primary reagent includes a proteinaceous capture reagent that specifically binds to a target molecule. In one aspect, the primary reagent includes a proteinaceous primary reagent selected from an antibody, an antigen, a receptor, an enzyme or a combination thereof. In one aspect, the primary reagent includes a capture-target hybrid that comprises a target molecule or target analyte. In one aspect, the primary reagent includes a capture-target hybrid selected from a cell, viral derivative, cellular organelle, subcellular structure, vesicle, therapeutic molecule or a combination thereof. In one aspect, the primary reagent includes an antigen-binding substance. In one aspect, the primary reagent includes an antibody or an antigen-binding fragment thereof. In one aspect, the primary reagent includes a first member of a binding pair and the target molecule includes a second member of the binding pair. In one aspect, the binding pair includes streptavidin or avidin and biotin.

In one aspect, the assay surface is part of a multi-well plate. In one aspect, the assay surface is a carbon-containing assay surface that is part of a multi-well plate. In one aspect, the carbon-containing assay surface includes an electrode. In one aspect, the carbon-containing assay surface is part of a multi-well plate and one or more wells of the multi-well plate include one or more electrodes. In one aspect, the carbon-containing assay surface is part of a multi-well plate and each well of the plate includes one or more electrodes. In one aspect, the carbon-containing assay surface includes a carbon-containing electrode. In one aspect, the carbon-containing assay surface includes a carbon-ink electrode.

In one aspect, the primary reagent is a proteinaceous primary reagent that includes an antibody or an antigen-binding antibody fragment and the secondary reagent includes a thiolated oligonucleotide. In one aspect, the primary reagent is a proteinaceous primary reagent that binds a target that is a surface protein of an extracellular vesicle (EV). In one aspect, the extracellular vesicle is an exosome. In one aspect, the target is an exosome-associated protein. In one aspect, the target is encapsulated by the exosome. In one aspect, the secondary reagent binds to a target that is an exosome-associate protein. In one aspect, the secondary regent binds to a target encapsulated by an exosome.

### BRIEF DESCRIPTION OF THE FIGURES

FIG. 1 is a graph showing assay sensitivity of a bifunctional assay surface prepared using a direct coating method described herein.

FIG. 2 is a graph showing assay sensitivity of a bifunctional assay surface prepared using an overcoating method described herein.

FIG. 3A compares assay sensitivity for assay plates with different types of anchor oligonucleotide: a biotinylated and pegylated anchor oligonucleotide attached using a direct coating method described herein (Bio-Peg-SH/Anchor); a thiolated anchor oligonucleotide (Anchor-SH); and no anchor oligonucleotide.

FIG. 3B compares nonspecific binding (NSB) for assay plates with different types of anchor oligonucleotide: a biotinylated and pegylated anchor oligonucleotide attached using a direct coating method described herein (Bio-Peg-SH/Anchor); a thiolated anchor oligonucleotide (Anchor-SH); and no anchor oligonucleotide.

FIG. 3C compares the ratio of signal to nonspecific binding for assay plates with different types of anchor oligonucleotide: a biotinylated and pegylated anchor oligonucleotide attached using a direct coating method described herein (Bio-Peg-SH/Anchor); a thiolated anchor oligonucleotide (Anchor-SH); and no anchor oligonucleotide.

FIG. 4 shows signal generation of FITC-Peg-SH immobilized by an overcoating method described herein.

FIG. 5A shows non-specific binding (NSB) in an assay for IFNg using an assay surface modified with Peg oligomers. Five Peg-SH reagents (MW 350, 550, 750, 1000, and 2000) were immobilized via overcoating and tested in standard sandwich format (10-plex).

FIG. 5B shows non-specific binding (NSB) in an assay for TNFα using an assay surface modified with Peg oligomers. Five Peg-SH reagents (MW 350, 550, 750, 1000, and 2000) were immobilized via overcoating and tested in standard sandwich format (10-plex).

### DETAILED DESCRIPTION OF THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

As used herein, the term “about” is used to indicate that a value includes the inherent variation of error for the device, or the method being employed to determine the value.

As used herein, “between” is a range inclusive of the ends of the range. For example, a number between x and y explicitly includes the numbers x and y, and any numbers that fall within x and y.

As used herein, “room temperature” is an indoor temperature suitable for long term storage of biological matter and laboratory experimentation, typically ranging between 15-28° C. In embodiments, room temperature is from 20-25° C.

In the context of analytes measured in an assay, or a reagents used in an assay, the term “plurality” means more than one structurally and/or functionally different analyte or reagent (e.g., reagent A and reagent B), rather than just more than one copy of the analyte or reagent (e.g., reagent A and another copy of reagent A). For example, the term “plurality of immobilized reagents,” means that more than one structurally or functionally different reagent is immobilized and does not describe a situation where there are multiple copies of one reagent. However, use of the term “plurality” in this context does not preclude the possibility that multiple copies are present of any of the plurality of analytes or reagents. For example, a plurality of immobilized reagents could refer to immobilized reagents that include one or more copies of reagent A and one or more copies of reagent B. In the context of an assay surface, the terms “bifunctional” or “multifunctional” refers to a surface on which two or more different types of reagents are immobilized. In one aspect, the different types of reagents are immobilized on the surface by different surface chemistries. As used herein, “surface chemistry” refers to the chemical reactions resulting in the immobilization of a reagent on an assay surface. Surface chemistry includes covalent and non-covalent interactions. Covalent immobilization refers to immobilization by the formation of one or more covalent bonds between reactive functional groups on the reagent and assay surface. Non-covalent interactions include, for example, hydrogen bonding, electrostatic or ionic interactions, and Van der Waals forces. Passive adsorption refers to immobilization of a biomolecule on a surface via hydrophobic interactions or hydrophobic and ionic interactions between the biomolecule and the surface.

As used herein, the term “polypeptide” is intended to encompass a singular polypeptide as well as plural polypeptides and refers to a molecule made up of amino acid monomers linked by peptide bonds. The term polypeptide refers to any chain or chains of amino acids with a peptide backbone and does not refer to a specific length of the product. Thus, peptides, dipeptides, tripeptides, oligopeptides, protein, amino acid chain, or any other term used to refer to a chain or chains of amino acids, are included within the definition of polypeptide, and the term polypeptide may be used instead of or interchangeably with any of these terms. The term polypeptide also includes products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology. A polypeptide is not necessarily translated from a designated nucleic acid sequence. It may be generated in any manner, including by chemical synthesis. In the context of polypeptides, a “sequence” refers to an order of amino acids in a polypeptide in an amino to carboxyl terminal direction in which residues that neighbor each other in the sequence are contiguous in the primary structure of the polypeptide. As used herein, the term “proteinaceous” refers to a naturally occurring or non-naturally occurring macromolecule that includes one or more polypeptide chains, including, but not limited to, peptides, proteins, antibodies, antigens, enzymes, receptors, or a fragment or portion thereof.

The term “oligonucleotide,” as used herein refers to short polymers of nucleic acids with a phosphate backbone such as deoxyribonucleic acid (DNA) or ribonucleic acid (RNA), locked nucleic acid (LNA), peptide nucleic acid (PNA), or a combination thereof. In one aspect, oligonucleotides have a length from about 5, 10, 15, 20 or 25 nucleotides and up to about 50, 75, 100 or 150 nucleotides, or about 5, 10, 15, 20, 25, 30, 35, 40, 45, 50, 75, 100 or 150 nucleotides. Oligonucleotides may be designed to specifically hybridize to DNA or RNA sequences, for example, for detecting a target nucleotide with sequence that is complementary to the nucleotide sequence of the oligonucleotide probe. Oligonucleotides may be single-stranded or double-stranded and may be obtained by methods, including, but not limited to, isolation from a biological sample, recombinant synthesis and chemical synthesis. The term “oligonucleotide” may include structural analogs that include non-naturally occurring modifications. For example, an oligonucleotide may include a chemical modification that links it to another substance such as a label or provides a reactive functional group that can be linked to another substance. The oligonucleotide can also include one or more non-natural nucleotide bases.

The term “reactive functional group” refers to an atom or associated group of atoms that can undergo a further chemical reaction, for example, to form a covalent bond with another functional group. Examples of reactive functional groups include, but are not limited to, amino, thiol, hydroxy, and carbonyl groups. In one aspect, the reactive functional group includes a reactive thiol group. In one aspect, the reactive functional group is used to immobilize a biomolecule onto a surface. In one aspect, the reactive functional group is used to append a label to biomolecule. Labels that can be linked to nucleotides or nucleic acids through these chemical modifications include, but are not limited to, detectable moieties such as biotin, haptens, fluorophores, and electrochemiluminescent (ECL) labels.

The terms “antibody” and “immunoglobulin” can be used interchangeably to refer to a biomolecule that is capable of specifically binding to an antigen. In most vertebrate animals, antibodies exist as dimers of two heavy (H) chains that are each paired with a light (L) chain. The N-termini of the heavy and light chains include a variable domain (V_{H} and V_{L}, respectively) that provide the antibody with its unique antigen-binding specificity. As used herein, the term “antibody” refers to a whole antibody molecule or an antigen-binding fragment thereof. The antibody or a fragment thereof can be naturally produced, or partially or wholly synthetically or recombinantly produced. An antigen-binding fragment refers to any antibody fragment that includes at least a portion of the variable region of the immunoglobulin molecule and retains the binding specificity of the full-length immunoglobulin. The term antibody includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, including, but not limited to, Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. A “binding reagent” refers to reagent characterized by an ability to preferentially bind to another substance, which may be referred to as the “binding partner.” The binding reagent and binding partner can be referred to as a “binding pair.” Examples of binding pairs include, but are not limited to, biotin and streptavidin or avidin; complementary oligonucleotides; hapten and hapten binding partner; antibody/antigen binding pairs; receptor/ligand binding pairs; and enzyme/substrate binding pairs. In one aspect, a first member of a binding pair is immobilized in a binding domain on an assay surface and a second member of a binding pair is a target.

The term “specifically binds” refers to the preferential interaction between a binding reagent and its target as compared to the interaction between the binding reagent and other molecules or components in a sample. Specific binding of between a binding reagent and a target is due to complementarity between the binding domain of the binding reagent and the target and non-covalent forces such as electrostatic forces, hydrogen bonds, Van der Waals forces and hydrophobic forces. As used herein, the term “preferentially binds,” means that one member of a binding pair binds to its binding partner under suitable conditions without any significant binding, for example, without any statistically significant binding, to other compounds present in a sample. In one aspect, members of a binding pair, for example, a binding reagent and its target, have an affinity for each other that is at least about 50-, 75-, or 100-fold greater than the affinity between either member of the binding pair and other compounds present in the sample.

The term “target” refers to a substance in a sample which can be specifically bound by a reagent, for example, to retain the target at a particular location on an assay surface. In one aspect, the target is retained at a particular location on an assay surface so that the presence or amount of target in a sample can be determined, in which case, the target can be referred to as a target analyte. In one aspect, a target is retained at a particular location on an assay surface to facilitate the detection of a different target analyte. In one aspect, the target covalently binds to a reagent immobilized on an assay surface. In one aspect, the target non-covalently binds to a reagent immobilized on an assay surface. In one aspect, the target binds directly to a reagent immobilized on an assay surface. In one aspect, the target is bound indirectly to a reagent immobilized on the assay surface, for example, via a binding member that binds to the target and is also bound by the reagent immobilized on the assay surface. Examples of targets include, but are not limited to, cells; cellular membranes; organelles; receptors, for example, receptors on vesicles, lipids, or cell membranes; ligands, agonists or antagonists which bind to a receptor; antibodies; antigens; proteins; peptides; polysaccharides; oligonucleotides or polynucleotides, including, for example, mRNA, tRNA, rRNA, DNA, cDNA, or an amplification product or amplicon; or a therapeutic molecule such as a drug. In one aspect, the target includes a member of a binding pair, such as biotin, avidin or streptavidin.

“Complementary” refers to nucleic acid molecules or a sequence of nucleic acid molecules that interact by the formation of hydrogen bonds, for example, according to the Watson-Crick base-pairing model, for example, in which A pairs with T or U; and C pairs with G. For example, hybridization can occur between two complementary DNA molecules (DNA-DNA hybridization), two RNA molecules (RNA-RNA hybridization), or between complementary DNA and RNA molecules (DNA-RNA hybridization). Hybridization can occur between a short nucleotide sequence that is complementary to a portion of a longer nucleotide sequence. Perfect complementarity or 100% complementarity refers to the situation in which each nucleotide of one oligonucleotides sequence or region can hydrogen bond with each nucleotide of at least a portion of second oligonucleotide strand or region. Hybridization can occur between sequences that do not have 100% “sequence complementarity” (i.e., sequences where less than 100% of the nucleotides align based on a base-pairing model such as the Watson-Crick base-pairing model). Generally, sequences having less sequence complementarity are less stable and less likely hybridize than sequences having greater sequence complementarity. In one aspect, the nucleotides of the complementary sequences have 100% sequence complementarity based on the Watson-Crick model. In another aspect, the nucleotides of the complementary sequences have at least about 90%, 95%, 96%, 97%, 98% or 99% sequence complementarity based on the Watson-Crick model and are able to hybridize under stringent hybridization conditions. It is understood that complementary sequences need not hybridize along their entire length, i.e., a shorter oligonucleotide sequence can hybridize to a portion of a longer oligonucleotide sequence to which is it complementary.

Whether or not two complementary sequences hybridize can depend on the stringency of the hybridization conditions, which can vary depending on conditions such as temperature, solvent, ionic strength and other parameters. The stringency of the hybridization conditions can be selected to provide selective formation or maintenance of a desired hybridization product of two complementary nucleic acid sequences, in the presence of other potentially cross-reacting or interfering sequences. Stringent conditions are sequence-dependent—typically longer complementary sequences selectively hybridize at higher temperatures than shorter complementary sequences.

As used herein, “capture molecule” refers to a molecule, or complex or combination thereof, that is capable of specifically binding to a target. In one aspect, the capture molecule is a peptide or protein, including, for example, an antibody, an antigen-binding antibody fragment or a receptor. In another aspect, the capture molecule is an oligonucleotide or nucleic acid that hybridizes to a complementary oligonucleotide sequence of a target under stringent hybridization conditions. In one aspect, the capture molecule includes a vitamin, oligosaccharide, carbohydrate, lipid, small molecule, or a complex thereof. In one aspect, the primary reagent includes a primary capture molecule that specifically binds to a primary target. In one aspect, the secondary reagent includes a secondary capture molecule that specifically binds to a secondary target. In one aspect, the secondary target is associated with the primary target. In one aspect, the secondary target is encapsulated by the primary target.

The term “biomolecule” as used herein refers to any compound or substance that is produced by a living organism, e.g., a cell. In one aspect, the biomolecule is isolated from biological material by solvent, thermal, and/or physical methods of extraction, separation, purification known in the art.

As used herein, a “capture-target hybrid” is a biomolecule, molecule or complex or combination thereof that comprises a region that renders it capable of being immobilized in a binding domain on an assay surface and comprises a target analyte. In one aspect, the target analyte is on the surface of the capture-target hybrid. In one aspect, the target analyte is on the surface of a biomolecule, e.g., a cell or organelle. In another aspect, the target is on the surface of an artificial or engineered capture molecule. In one aspect, the capture-target hybrid comprises an antigen-binding substance.

In one aspect, the capture-target hybrid is a cell, virus derivative, cellular organelle, subcellular structure, vesicle or therapeutic molecule. In one aspect, the capture-target hybrid is a cell, for example, a bacterial cell, an archaeal cell, a mammalian cell, an insect cell or a plant cell. In one aspect, the capture-target hybrid is a cell engineered to produce a surface protein analyte or other desired target analyte. In one aspect, the capture-target hybrid is a cellular organelle, for example, a nucleus, endoplasmic reticulum, Golgi apparatus, mitochondria, vacuole, chloroplast, inflammasome, cellular translation complex and combinations thereof. In one aspect, the capture-target hybrid is a viral derivative, for example, a virus, a viral particle, a virion, a virion membrane, a virion membrane fragment and combinations thereof. In one aspect, the capture-target hybrid is a subcellular structure, for example, a micelle, membrane preparation, membrane raft, membrane ghost, membrane vesicle, membrane fragment, artificial lipid membrane, or combinations thereof. In one aspect, the capture-target hybrid is a vesicle such as a lysosome, endosome, peroxisome, liposome and combinations thereof.

A “viral derivative” as used herein refers to particles or fragments obtained from or derived from a virus. The viral derivative can be formed by disassembly through mechanical manipulation, intracellular factors and molecular mechanisms including receptor-induced structural remodeling, low-pH-activated conformational change, protease-dependent proteolysis, reductase-catalyzed disulfide bond disruption, and chaperone-mediated unfolding/refolding reactions that viruses use to drive their disassembly. In one aspect, the viral derivative includes a virus, a viral particle, a virion, a virion membrane, and a virion membrane fragment.

“Subcellular structure” refers to compartments or structures that are situated or occurring within a whole cell. In one aspect, the subcellular structure can be derived from compartments or structures in a whole cell or artificially prepared by various means known in the art. The subcellular structure can be separated or removed from the cell by cell fractionation methods including extraction, homogenization and centrifugation techniques known in the art. In one aspect, the subcellular structure includes a micelle, membrane preparation, membrane raft, membrane ghost, membrane vesicle, membrane fragment, artificial lipid membrane, or combinations thereof.

As used herein, the term “vesicle” refers to a structure within or outside a cell, consisting of liquid or cytoplasm enclosed by a lipid bilayer. The vesicle can be separated or removed from the cell by cell fractionation methods including extraction, homogenization and centrifugation techniques known in the art. In one aspect, the vesicle is artificial or synthetic vesicles prepared in vitro using biochemical and microfluidic synthesis techniques known in the art. In one aspect, the vesicle includes a lysosome, endosome, peroxisome, liposome and combinations thereof.

### Overview

Methods for immobilizing reagents on a surface are provided herein. In one aspect, the surface is an assay surface used for conducting assays to detect and/or quantify one or more analytes of interest in a sample. In one aspect, one or more reagents are immobilized on the assay surface. In one aspect, the surface is a bifunctional or multifunctional assay surface in which two or more reagents with differing structure and/or function are immobilized using different surface chemistries. In one aspect, the surface is a bifunctional or multifunctional assay surface in which a primary reagent and a secondary reagent are immobilized. In one aspect, the primary reagent and the secondary reagent have a different structure and/or function. For example, in one aspect the primary reagent is a proteinaceous reagent and the secondary reagent is a thiol-containing reagent. In one aspect the primary reagent is a proteinaceous reagent and the secondary reagent is a thiolated oligonucleotide. In one aspect, the primary reagent and the secondary reagent are immobilized using different surface chemistries. In one aspect, the assay surface is a multi-well plate. In one aspect, the assay surface includes particles or beads.

Biomolecules can be attached to surfaces by several mechanisms. Passive adsorption refers to immobilization via hydrophobic interactions or hydrophobic and ionic interactions between the biomolecule and the surface. In one aspect, one or more reagents are immobilized on an assay surface by passive adsorption. In one aspect, a plurality of reagents are immobilized on an assay surface by passive adsorption. In one aspect, one or more primary reagents are immobilized on an assay surface by passive adsorption. In one aspect, a plurality of primary reagents are immobilized on an assay surface by passive adsorption. In one aspect, one or more proteinaceous primary reagents or capture-target hybrids are immobilized on an assay surface by passive adsorption. In one aspect, a plurality of proteinaceous primary reagents or capture-target hybrids are immobilized on an assay surface by passive adsorption. In one aspect, the proteinaceous primary reagent is a capture molecule. In one aspect, the proteinaceous primary reagent includes bovine serum albumin (BSA), for example, the proteinaceous primary reagent can include an oligonucleotide conjugated to BSA. In one aspect, a member of a binding pair is immobilized on an assay surface by passive adsorption. In one aspect, streptavidin is immobilized on the assay surface by passive adsorption. In one aspect, passive adsorption is enhanced by using a slightly ionic, hydrophobic surface.

Covalent immobilization refers to immobilization by the formation of one or more covalent bonds between one or more reactive functional groups on the reagent and one or more reactive functional groups on the assay surface. The term “reactive functional group” refers to an atom or associated group of atoms that can undergo a further chemical reaction, for example, to form a covalent bond with another functional group. Examples of reactive functional groups include, but are not limited to, amino, thiol, hydroxy, and carbonyl groups. In one aspect, the reactive functional group includes a thiol group. In one aspect, a reagent is covalently immobilized on an assay surface through a covalent bond formed between a thiol moiety (—SH) on the reagent and a reactive functional group on the assay surface. In one aspect, an oligonucleotide is covalently immobilized on an assay surface through a covalent bond formed between a thiol moiety on the oligonucleotide and a reactive functional group on the assay surface.

In some instances, it may be desirable to prepare a bifunctional or multifunctional assay surface on which two or more reagents with differing structure and/or function are immobilized. For example, PCT Publication No. WO 2014/165061, filed Mar. 12, 2014, and entitled IMPROVED ASSAY METHODS, the disclosure of which is hereby incorporated by reference herein in its entirety, describes methods for amplifying a signal in an immunoassay using a bifunctional assay surface on which a primary capture reagent and a secondary anchoring reagent are immobilized. In one aspect, the primary capture reagent is a proteinaceous primary reagent such as an antibody, an antigen-binding antibody fragment or receptor and the secondary anchoring reagent is an oligonucleotide. In one aspect, the primary capture reagent is a capture-target hybrid such as a cell, virus, cellular organelle, subcellular structure, vesicle or therapeutic molecule and the secondary anchoring reagent is an oligonucleotide. In one aspect, the primary capture reagent and secondary anchoring reagent are dispensed on the assay surface at the same time and immobilized using the same surface chemistry for both reagents. For example, the secondary oligonucleotide reagent can be conjugated to a protein such as bovine serum albumin (BSA) such that both the proteinaceous primary reagent and the secondary oligonucleotide anchor reagent are immobilized by passive adsorption. Alternately, the secondary oligonucleotide anchor reagent and the primary capture reagent can be conjugated to a hapten such as biotin and immobilized on a streptavidin coated surface. Although the bifunctional assay surface described in PCT Publication No. WO 2014/165061 provides increased assay sensitivity, the assay module must be specially prepared for a particular application, which can increase production costs.

Provided herein are methods for preparing a bifunctional assay surface in which a primary reagent and a secondary reagent are immobilized on an assay surface. In one aspect, the primary reagent and the secondary reagent are immobilized on the assay surface using different surface chemistries. In one aspect, the primary reagent is immobilized on the assay surface by passive adsorption and the secondary reagent is immobilized by the formation of a covalent bond between a reactive functional group on the secondary reagent and a reactive functional group on the assay surface. In one aspect, the primary reagent is immobilized on the assay surface through the interaction of a binding pair. In one aspect, the primary reagent includes a biotin moiety and is immobilized on a streptavidin coated surface through the interaction of the streptavidin/biotin binding pair. In one aspect, the primary reagent is a proteinaceous molecule such as an antibody, an antigen-binding antibody fragment or a receptor and is immobilized on the assay surface by passive adsorption.

In one aspect, a “direct” coating method is provided in which a primary and a secondary reagent are immobilized on an assay surface simultaneously using different surface chemistries. In another aspect, a “direct” coating method is provided in which proteinaceous streptavidin and a thiol-modified anchor oligonucleotide are immobilized to the plate surface. Advantageously, the secondary reagent does not need to be modified so that it can be immobilized using the same surface chemistry as the primary reagent. For example, a proteinaceous primary reagent or capture-target hybrid can be immobilized by passive adsorption, or through a binding pair such as avidin/streptavidin and biotin at the same time as an oligonucleotide secondary reagent, for example, a thiolated oligonucleotide secondary reagent, without the need to conjugate the oligonucleotide secondary reagent to a protein such as bovine serum albumin (BSA) or a member of a binding pair such as biotin.

In another aspect, an “overcoating” method is provided in which a secondary reagent can be immobilized onto an assay surface on which a primary reagent is already immobilized. Advantageously, a secondary reagent can be immobilized on an existing assay surface on which a primary reagent was previously immobilized.

As shown herein, assay surfaces prepared by the overcoating and direct coating methods have equivalent or better performance as compared to bifunctional surfaces in which the different reagents are immobilized using the same surface chemistries.

### Assay Module

In one aspect, a method is provided for preparing a bifunctional surface. In one aspect, a method is provided for preparing a bifunctional surface in an assay module. In one aspect, the assay module includes a plurality of surfaces, including, for example, particles or beads. In one aspect, the assay module includes a unitary surface, such as a cartridge or a plate. In one aspect, the assay module includes one or more assay cells, such as wells, compartments, chambers, conduits, or flow cells. In one aspect, the assay module is a multi-well assay plate. Multi-well assay plates are available in a variety of forms, sizes, and shapes, with standards dimensions used for high-throughput assays. In one aspect, the assay module is a multi-well plate with a standard well configuration, for example, a 6-well, 24-well, 96-well, 384-well, 1536-well, 6144-well or 9600-well plate. In one aspect, the assay module is a 96-well plate.

In one aspect, the assay surface includes a two-dimensional patterned array in which one or more reagents can be printed at known locations, referred to as binding domains. In one aspect, the assay surface includes a patterned array of discrete, non-overlapping, addressable binding domains to which a plurality of reagents are immobilized, wherein the identity of the reagent in each binding domain is known and can be correlated with a target. In one aspect, at least two of the binding domains include reagents with different binding specificities from each other. In one aspect, the array is arranged in a symmetrical grid pattern. In other aspects, the array is arranged another pattern, including, but not limited to, radially distributed lines, spiral lines, or ordered clusters. In another aspect, each binding domain is positioned on a surface of one or more microparticles or beads wherein the microparticles or beads are coded to allow for discrimination between different binding domains.

In one aspect, a primary reagent is immobilized on an assay surface in an array. In one aspect, a proteinaceous primary reagent or capture-target hybrid is immobilized on an assay surface in an array. In one aspect, proteinaceous streptavidin and a thiol-modified anchor oligonucleotide are immobilized on an assay surface in an array. In one aspect, the primary reagent is immobilized in an array on a carbon-containing assay surface. In one aspect, proteinaceous streptavidin and a thiol-modified anchor oligonucleotide are immobilized in an array on a carbon-containing assay surface. In one aspect, the primary reagent is immobilized in an array on a carbon-containing electrode. In one aspect, proteinaceous streptavidin and a thiol-modified anchor oligonucleotide are immobilized in an array on a carbon-containing electrode. In one aspect, the secondary reagent is immobilized in an array. In one aspect, the assay surface is coated with the secondary reagent.

In one aspect, the assay surface includes one or more binding domains. In one aspect, the assay surface includes a plurality of binding domains. In one aspect, the assay module is a multi-well plate and one or more wells of a multi-well plate include one or more binding domains. In one aspect, each well of a multi-well plate includes a plurality of binding domains. In one aspect, each well includes at least 4, 5, 6, 7, 8, 9, 10, 11, 12, 13, 14, or 15 binding domains and up to 25, 64, 100 or 250 binding domains. In one aspect, each well includes 4, 7, 10, 25, 64, or 100 binding domains. In one aspect, at least two of the binding domains include two different reagents.

In one aspect, the assay module includes discrete binding domains on one or more solid surfaces. In one aspect, the assay module includes one or more particles or beads and the one or more particles or beads include one or more binding domains. In one aspect, each binding domain is on a separate surface, for example, on the surface of a separate bead. In one aspect, the assay module includes one or more, or a plurality of particles or beads on which one or more primary reagents and one or more secondary reagents are immobilized. In one aspect, the assay module includes one or more, or a plurality of particles or beads on which one or more proteinaceous primary reagents and one or more thiol-containing secondary reagents are immobilized. In one aspect, the assay module includes one or more, or a plurality of particles or beads on which one or more capture-target hybrids and one or more thiol-containing secondary reagents are immobilized. In one aspect, the binding domains are the individual beads, such that discrete assay signals are generated on and measured from each binding domain. In one aspect, the assay module includes one or more, or a plurality of coded particles or beads on which one or more primary capture reagents and one or more secondary reagents are immobilized, wherein the coding identifies the capture reagent and target for a specific bead.

In one aspect, one or more binding domains on the assay surface include one or more assay reagents. In one aspect, each binding domain on the assay surface includes one or more assay reagents. In one aspect, one or more binding domains include a primary reagent. In one aspect, the primary reagents in a particular binding domain all have the same binding specificity and the primary reagents in one binding domain have a different binding specificity than the primary reagents in another binding domain. In one aspect, one or more binding domains include a secondary reagent. In one aspect, the secondary reagents in a particular binding domain all have the same binding specificity and the secondary reagents in one binding domain have a different binding specificity than the secondary reagents in another binding domain. In one aspect, the secondary reagents in more than one binding domain have the same binding specificity.

In one aspect, a primary reagent is immobilized in a first binding domain and a secondary reagent is immobilized in a second binding domain. In one aspect, a plurality of primary reagents with different binding specificities are immobilized in a plurality of first binding domains and a plurality of secondary reagents with different binding specificities are immobilized in a plurality of second binding domains.

In one aspect, one binding domain in an array may include a different primary reagent than another binding domain, such that some or all of the binding domains in the array include a “unique” primary reagent. In one aspect, one binding domain in an array may include a different secondary reagent than another binding domain, such that some or all of the binding domains in the array include a “unique” secondary reagent. In one aspect, each binding domain in an array includes the same secondary reagent, such that some or all of the binding domains in the array include a “common secondary reagent.” In one aspect, one or more binding domains include a primary reagent and a secondary reagent. In one aspect, one or more binding domains include a unique primary reagent and a unique secondary reagent. In one aspect, one or more binding domains include a unique primary reagent and a common secondary reagent.

In one aspect, one or more binding domains of the assay surface include a proteinaceous primary reagent or capture target hybrid. In one aspect, one or more binding domains of the assay surface include a thiol-containing secondary reagent. In one aspect, a proteinaceous primary reagent or capture-target hybrid is immobilized in a first binding domain that does not include thiol-containing secondary reagent and a thiol-containing secondary reagent is immobilized in a second binding domain that does not include primary reagent. In one aspect, each binding domain includes a proteinaceous primary reagent or capture-target hybrid. In one aspect, each binding domain includes a thiol-containing secondary reagent. In one aspect, one or more binding domains on the assay surface include a proteinaceous primary reagent and a thiol-containing secondary reagent. In one aspect, one or more binding domains on the assay surface include a capture-target hybrid and a thiol-containing secondary reagent. In one aspect, each binding domain on the assay surface includes a proteinaceous primary reagent and a thiol-containing secondary reagent. In one aspect, each binding domain on the assay surface includes a capture-target hybrid and a thiol-containing secondary reagent. In one aspect, each binding domain includes a unique proteinaceous primary reagent and a unique thiol-containing secondary reagent. In one aspect, each binding domain includes a unique capture-target hybrid and a unique thiol-containing secondary reagent. In one aspect, each binding domain includes a unique proteinaceous primary reagent and a common thiol-containing secondary reagent. In one aspect, each binding domain includes a unique capture-target hybrid and a common thiol-containing secondary reagent.

In one aspect, the assay surface includes a multi-well plate and a plurality of primary reagents are immobilized in one or more binding domains on the surface of the multi-well plate. In one aspect, one or more binding domains on the surface of the well of the multi-well plate includes one or more copies of a proteinaceous primary reagent or capture-target hybrid. In one aspect, the proteinaceous primary reagents immobilized in a particular binding domain all have the same binding specificity and the proteinaceous primary reagents immobilized in one binding domain have a different binding specificity than the proteinaceous primary reagents immobilized in another binding domain. In one aspect, the capture-target hybrids immobilized in a particular binding domain all have the same binding specificity and the capture-target hybrids immobilized in one binding domain have a different binding specificity than the capture-target hybrids immobilized in another binding domain. In one aspect, the assay surface includes a multi-well plate and one or more thiol-containing reagents are immobilized in one or more binding domains on the surface of the multi-well plate. In one aspect, each binding domain includes one or more copies of a thiol-containing secondary reagent. In one aspect, a proteinaceous primary reagent or capture-target hybrid is immobilized in a first binding domain and a thiol-containing secondary reagent is immobilized in a second binding domain. In one aspect, each binding domain on the surface of the well of a multi-well plate includes a primary reagent and a secondary reagent. In one aspect, each binding domain includes one or more copies of a unique thiol-containing secondary reagent. It is noted that the “unique” thiol-containing secondary reagent in one well of a multi-well plate may be included in other wells of the multi-well plate. In one aspect, all binding domains within a well of the multi-well plate include one or more copies of a common thiol-containing secondary reagent. It is noted that other wells of the multi-well plate may include the same thiol-containing secondary reagent or a different thiol-containing secondary reagent. In one aspect, each binding domain includes a unique proteinaceous primary reagent or capture-target hybrid and a unique thiol-containing secondary reagent. In one aspect, each binding domain includes a unique proteinaceous primary reagent or capture-target hybrid and a common thiol-containing secondary reagent. In one aspect, each binding domain includes a unique capture antibody or antigen-binding antibody fragment and a unique thiolated oligonucleotide. In one aspect, each binding domain includes a unique capture antibody or antigen-binding antibody fragment and a common thiolated oligonucleotide.

In one aspect, proteinaceous streptavidin and a thiol-modified anchor oligonucleotide are immobilized on an assay surface in an array. In one aspect, the proteinaceous streptavidin is immobilized by passive adsorption of the streptavidin to the plate surface and the thiol-modified anchor oligonucleotide is immobilized by covalent bonding of the thiol-group the plate surface. In one aspect, proteinaceous streptavidin and a thiol-modified anchor oligonucleotide are immobilized in an array on a carbon-containing assay surface. In one aspect, proteinaceous streptavidin and a thiol-modified anchor oligonucleotide are immobilized in an array on a carbon-containing electrode.

In one aspect, a proteinaceous primary reagent or capture-target hybrid is immobilized on an assay surface in an array. In one aspect, the proteinaceous primary reagent or capture-target hybrid is immobilized in an array on a carbon-containing assay surface. In one aspect, the proteinaceous primary reagent or capture-target hybrid is immobilized in an array on a carbon-containing electrode.

In one aspect, a proteinaceous primary reagent and a thiol-containing secondary reagent are immobilized on an assay surface in an array. In one aspect, the proteinaceous primary reagent and thiol-containing secondary reagent are immobilized in an array on a carbon-containing assay surface. In one aspect, the proteinaceous primary reagent and thiol-containing secondary reagent are immobilized in an array on a carbon-containing electrode. In one aspect, a capture-target hybrid and a thiol-containing secondary reagent are immobilized on an assay surface in an array. In one aspect, the capture-target hybrid and thiol-containing secondary reagent are immobilized in an array on a carbon-containing assay surface. In one aspect, the capture-target hybrid and thiol-containing secondary reagent are immobilized in an array on a carbon-containing electrode.

In one aspect, a primary reagent is immobilized on the assay surface through a different surface chemistry than a secondary reagent. In one aspect, the primary reagent is a proteinaceous primary reagent. In one aspect, the primary reagent is a proteinaceous capture reagent. In one aspect, the primary reagent is a capture-target hybrid. In one aspect, the proteinaceous primary reagent or capture-target hybrid is immobilized on the assay surface by passive adsorption. In one aspect, the proteinaceous primary reagent or capture-target hybrid is immobilized on the assay surface through a binding partner, such as streptavidin or avidin and biotin. In one aspect, the proteinaceous primary reagent or a capture-target hybrid includes a biotin moiety and the assay surface is coated with streptavidin. In one aspect, the secondary reagent is a thiol-containing secondary reagent. In one aspect, the proteinaceous primary reagent or capture-target hybrid is immobilized on the assay surface through a different surface chemistry than the thiol-containing secondary reagent. In one aspect, the secondary reagent is immobilized on the assay surface through the interaction of the thiol group and a reactive functional group on the assay surface. In one aspect, the secondary reagent is immobilized on the assay surface by covalent bonding between the thiol group and a reactive functional group on the assay surface. In one aspect, the thiol-containing secondary reagent is immobilized on the assay surface through the formation of a disulfide bond or maleimide linkage. In one aspect, one or more thiol-containing secondary reagents are immobilized on a carbon-containing assay surface. In one aspect, the assay surface is modified with a thiol-reactive moiety such as a maleimide, an iodosuccinimide or an activated disulfide (such as a pyridyldisulfide).

In one aspect, the proteinaceous primary reagent is a proteinaceous capture reagent. As used herein, a “capture reagent” is a reagent that is immobilized on an assay surface that binds to a target that may be present in a sample. In one aspect, the capture reagent specifically binds to a target. In one aspect, the primary capture reagent specifically binds to a primary target. In one aspect, the proteinaceous primary reagent includes an antigen-binding substance. In one aspect, the proteinaceous primary reagent includes an antibody or an antigen-binding antibody fragment. In one aspect, the proteinaceous primary reagent is an enzyme. In one aspect, the proteinaceous capture reagent is a receptor. In one aspect, the proteinaceous capture reagent includes a proteinaceous portion and a member of a binding pair, in which a first member of the binding pair is attached to the proteinaceous primary reagent and the other member of the binding pair is attached to a target. Non-limiting examples of binding pairs include biotin and streptavidin or avidin; complementary oligonucleotides; hapten and hapten binding partner; receptor/ligand; enzyme/substrate and antibody/antigen binding pairs. In one aspect, proteinaceous primary reagent includes a proteinaceous portion and the binding pair is selected from: streptavidin or avidin and biotin. In one aspect, the primary reagent includes a target binding portion and a proteinaceous portion. In one aspect, the proteinaceous portion of the primary reagent includes bovine serum albumin (BSA). In one aspect, the target binding portion of the primary reagent includes a capture molecule. In one aspect, the capture molecule is a proteinaceous molecule, such as a peptide or protein, including, for example, an antibody, an antigen-binding antibody fragment or an antigen; a receptor or a ligand; or an enzyme or a substrate. In another aspect, the capture molecule includes an oligonucleotide or nucleic acid that hybridizes to a complementary oligonucleotide sequence of a target under stringent hybridization conditions. In one aspect, the capture molecule includes a vitamin, oligosaccharide, carbohydrate, lipid, small molecule, or a complex thereof.

In one aspect, the reactive functional group is a thiol group. As used herein, a “thiol-containing secondary reagent” refers to a reagent that includes a sulfhydryl moiety (—SH). In one aspect, a thiol-containing secondary reagent is covalently immobilized on the surface through a reactive functional group. In one aspect, the thiol-containing secondary reagent is an oligonucleotide, aptamer, aptamer ligand, antibody, antigen, ligand, receptor, hapten, epitope, a mimotope, or a combination or complex thereof. In one aspect, the thiol-containing secondary reagent includes a thiol-modified single stranded or double stranded oligonucleotide, such as DNA or RNA. In one aspect, the thiol-containing secondary reagent includes a DNA-binding protein.

In one aspect, the thiol-containing secondary reagent includes a target binding portion and a thiolated portion, wherein the target binding portion of the secondary reagent is immobilized to the assay surface through the thiolated portion. In one aspect, the thiol-containing secondary reagent includes a target binding portion and a thiolated oligonucleotide, wherein the target binding portion of the secondary reagent is immobilized to the assay surface through the thiolated oligonucleotide. In one aspect, the target binding portion of the secondary reagent includes a capture molecule. In one aspect, the capture molecule is a peptide or protein, including, for example, an antibody, an antigen-binding antibody fragment or a receptor. In another aspect, the capture molecule is an oligonucleotide or nucleic acid that hybridizes to a complementary oligonucleotide sequence of a target under stringent hybridization conditions. In one aspect, the capture molecule includes a vitamin, oligosaccharide, carbohydrate, lipid, small molecule, or a complex thereof.

In one aspect, the secondary reagent includes an oligonucleotide portion that hybridizes to a complementary oligonucleotide under stringent conditions. In one aspect, the complementary oligonucleotide is a target analyte. In one aspect, the complementary oligonucleotide is a targeting oligonucleotide that is associated with a target analyte, for example, by covalent or non-covalent interactions, such that the target analyte can be immobilized on the assay surface via the interactions between the thiolated oligonucleotide and the targeting oligonucleotide. In one aspect, the target is a complementary oligonucleotide that is an amplification product or amplicon. In one aspect, the complementary oligonucleotide is an amplification product generated by an amplification technique, including, but not limited to, PCR (Polymerase Chain Reaction), LCR (Ligase Chain Reaction), SDA (Strand Displacement Amplification), 3SR (Self-Sustained Synthetic Reaction), and isothermal amplification methods, e.g., helicase-dependent amplification and rolling circle amplification (RCA).

In one aspect, the thiolated oligonucleotide has a length from about 5, 6, 7, 8, 9 or 10 nucleotides and up to about 20, 30, 40, 50, 75 or 100 nucleotides, or from about 5 to about 100, about 5 to about 50, or about 10 to about 30 nucleotides, or about 5, 6, 7, 8, 9, 10, 20, 30, 40, 50, 75 or 100 nucleotides. In one aspect, the thiolated oligonucleotide includes an oligonucleotide attached to a thiol group through a linker. In one aspect, the linker includes from about 3 to about 20 atoms or molecules or units, or at least about 3, 4, 5, 6, 7, 8, 9, 10 and up to about 10, 11, 12, 13, 14, 15, 16, 17, 18, 19 or 20 atoms or molecules or units. In one aspect, the thiolated oligonucleotide includes an oligonucleotide sequence having a 5′- and a 3′-end and a thiol group incorporated at the 5′ end (a 5′-terminal thiolated oligonucleotide), at the 3′ end (a 3′-terminal thiolated oligonucleotide), at an internal position of the oligonucleotide, or a combination thereof. In one aspect, the thiolated oligonucleotide includes deoxyribonucleic acid (DNA), ribonucleic acid (RNA), locked nucleic acid (LNA), peptide nucleic acid (PNA), or a combination thereof. In one aspect, the thiolated oligonucleotide includes one or more non-natural nucleotide bases. In one aspect, the non-natural nucleotide base is selected from: 2,6-Diaminopurine (2-Amino-dA); 5-Methyl deoxycytidine, Super T (5-hydroxybutynl-2′-deoxyuridine); or a combination thereof.

In one aspect, the thiol-containing secondary reagent includes a protein. In one aspect, the thiol-containing secondary reagent includes a protein such as bovine serum albumin (BSA). In one aspect, the thiol-containing secondary reagent includes a protein such BSA and is immobilized on the assay surface to reduce non-specific binding (NSB). Although sulfhydryl groups are present in many proteins, they can also be generated by reduction of native disulfide bonds, or be introduced by reacting primary amines with sulfhydryl-addition reagents, such as 2-iminothiolane (Traut's Reagent), SATA, SATP, SAT(PEG)₄, or a combination thereof.

In one aspect, the thiol-containing secondary reagent includes a thiolated member of a binding pair. In one aspect, the thiol-containing secondary reagent includes thiolated biotin (Thiol-Biotin). In one aspect, the thiol-containing secondary reagent includes thiolated polyethylene glycol (Thiol-PEG). In one aspect, the thiolated polyethylene glycol further includes biotin (Thiol-PEG-Biotin). In one aspect, the thiolated biotin is immobilized on an assay surface such that the biotin moiety can be used to immobilize additional reagents on the assay surface. In one aspect, streptavidin or avidin is conjugated to one or more assay reagents which are then immobilized onto the assay surface through the immobilized biotin moiety.

In one aspect, the thiol containing reagent includes one or more reactive groups. In one aspect, the thiol-containing secondary reagent includes an amine-reactive moiety. In one aspect, the thiol-containing secondary reagent includes fluorescein (FITC). In one aspect, the thiol-containing secondary reagent includes fluorescein and thiol heterofunctionalized polyethylene glycol (FITC-PEG-SH).

In one aspect, the assay module includes one or more electrodes. In one aspect, the assay surface includes an electrode surface. In one aspect, the electrode surface is a component of a multi-well plate. In one aspect, the electrode surface is a component of a particle or bead. In one aspect, the electrode is formed from a conductive material, including, but not limited to, metals such as gold, silver, platinum, nickel, steel, iridium, copper, aluminum, or a conductive alloy. In one aspect, the electrode includes an oxide coated metals, including, but not limited to, aluminum oxide coated aluminum. In one aspect, the electrode is constructed from a carbon-based material such as carbon, carbon black, graphitic carbon, carbon nanotubes, carbon fibrils, graphite, graphene, carbon fibers or a mixture thereof. In one aspect, the electrode is formed from elemental carbon, such as graphitic, carbon black, or carbon nanotubes. In one aspect, the electrode includes conducting carbon-polymer composites, conducting particles dispersed in a matrix such as carbon inks, carbon pastes, metal inks and graphene inks, and/or conducting polymers. In one aspect, the assay module is a multi-well plate with one or more carbon-containing electrodes, for example, one or more electrodes include carbon layers, and/or screen-printed layers of carbon inks.

In one aspect, the assay module is a multi-well plate. In one aspect, the assay module is a multi-well plate suitable for electrode induced luminescence-based assays. In one aspect, the assay surface is located within one or more wells of the multi-well plate. In one aspect, the assay module includes a plurality of wells and one or more electrodes. In one aspect, the assay module includes one or more working electrodes and one or more counter electrodes. In one aspect, one or more wells of the assay module include one or more electrodes. In one aspect, each well of the multi-well plate includes at least one working electrode and at least one counter electrode. In one aspect, one or more electrodes include one or more or a plurality of binding domains. In one aspect, the method includes detecting at least one electrochemiluminescent moiety to detect and/or quantify a target analyte in a sample. In one aspect, the electroluminescent moiety is an electrochemiluminescent label. In one aspect, the assay module is used for detection and/or quantification of a plurality of analytes in parallel. In one aspect, the assay module is used in a simultaneous multiplexed assay. Multiplexed measurement of analytes on a surface including a plurality of binding domains using electrochemiluminescence are known. See, for example, Meso Scale Diagnostics, LLC, MULTI-ARRAY® and SECTOR® Imager line of products and U.S. Pat. Nos. 7,842,246 and 6,977,722, the disclosures of which are incorporated herein by reference in their entireties.

### Target

Targets include, but are not limited to proteins, toxins, nucleic acids, amplification products or amplicons, microorganisms, viruses, cells, fungi, spores, carbohydrates, lipids, glycoproteins, lipoproteins, liposomes, exosomes, polysaccharides, drugs, hormones, steroids, nutrients, metabolites and any modified derivative of the above molecules, or any complex including one or more of the above molecules or combinations thereof. In one aspect, the target is an analyte of interest in a sample that is indicative of a disease or disease condition. In one aspect, the target is an analyte of interest in a sample that indicates whether the patient was exposed to that analyte. In one aspect, a target is retained on an assay surface to facilitate the detection of a different target that is an analyte of interest.

In one aspect, the primary reagent specifically binds to a target. In one aspect, the primary reagent specifically binds to a target analyte. In one aspect, the secondary reagent specifically binds to a target. In one aspect, the secondary reagent specifically binds to a target analyte. In one aspect, the primary reagent and the secondary reagent bind to a different target from each other. In one aspect, the primary reagent binds a target to facilitate detection of a target analyte bound by the secondary reagent. In one aspect, the secondary reagent binds a target to facilitate detection of a target analyte bound by the primary reagent.

In one aspect, the primary reagent is a proteinaceous capture reagent, such as an antibody or an antigen-binding antibody fragment and the thiol-containing secondary reagent includes a thiolated anchoring oligonucleotide. In one aspect, the primary reagent is a capture-target hybrid, such as a cell, subcellular structure, viral derivative, vesicle or therapeutic molecule and the thiol-containing secondary reagent includes a thiolated anchoring oligonucleotide. In one aspect, the capture antibody specifically binds to a target analyte. In one aspect, the capture-target hybrid comprises a target molecule or target analyte. In one aspect, the anchoring oligonucleotide reagent hybridizes with a target that includes an oligonucleotide. In one aspect, the anchoring oligonucleotide reagent hybridizes with a target that includes an oligonucleotide amplification product. In one aspect, the oligonucleotide amplification product is generated by an amplification technique, including, but not limited to, PCR (Polymerase Chain Reaction), LCR (Ligase Chain Reaction), SDA (Strand Displacement Amplification), 3SR (Self-Sustained Synthetic Reaction), and isothermal amplification methods, e.g., helicase-dependent amplification and rolling circle amplification (RCA). In one aspect, the amplification product includes an RCA amplicon. See, for example, PCT Publication No. WO 2014/165061, filed Mar. 12, 2014, and entitled IMPROVED ASSAY METHODS, the disclosure of which is hereby incorporated by reference herein in its entirety.

In one aspect, the primary reagent binds to a target associated with an extracellular vesicle (EV). In one aspect, the primary reagent binds to a surface protein of an extracellular vesicle. In one aspect, the primary reagent binds to an exosome. In one aspect, the exosome contains a signaling molecule including, but not limited to, a surface-bound or cytosolic protein, lipid, mRNA, miRNA, or a combination thereof. In one aspect, the identity and concentration of signaling molecules in an exosome is used to deduce its cellular origin and function. See, for example, International Application No. PCT/US2020/20288, filed Feb. 28, 2020, entitled IMMUNOASSAY METHODS, the disclosure of which is hereby incorporated by reference herein in its entirety.

In one aspect, the primary reagent specifically binds to a primary target and the secondary reagent specifically binds to a secondary target, for example, a target analyte associated with, or encapsulated by the primary target. In one aspect, the primary reagent specifically binds to a target that includes a surface protein of an extracellular vesicle (EV). In one aspect, the primary reagent is a proteinaceous primary reagent that binds a surface protein of an extracellular vesicle (EV). In one aspect, the extracellular vesicle is an exosome. In one aspect, the target analyte is an EV-associated protein. In one aspect, the target analyte is encapsulated by the EV. In one aspect, the secondary reagent binds to a target analyte that is an EV-associated protein. In one aspect, the secondary regent binds to a target analyte encapsulated by an EV.

### Capture-Target Hybrid

In embodiments, the capture-target hybrid is a biological moiety, for example, a cell, viral derivative, organelle, subcellular structure, vesicle, therapeutic molecule or combinations thereof, wherein the target and capture are integrated and together to form a capture-target hybrid. An example capture-target hybrid is a whole cell, whereby a portion of the cell binds to a plate and the target is on the surface of the cell, e.g., a protein. In one aspect, the capture-target hybrid is biotinylated and binds to streptavidin plates. In one aspect, the capture-target hybrid is coated with a thiol-containing compound and binds to streptavidin plates. In one aspect, the capture-target is uncoated and in its natural state or condition and binds to streptavidin plates. In one aspect, the capture-target hybrid is immobilized along with an anchoring oligonucleotide. Non-limiting examples of an anchoring oligonucleotide include a BSA-conjugated anchor oligonucleotide (BSA-oligo) or a thiol modified anchor oligonucleotide (Thiol-oligo). In one aspect, the capture-target hybrid and anchoring oligonucleotide are immobilized sequentially. Non-limiting examples of an anchoring oligonucleotide include a BSA-conjugated anchor oligonucleotide (BSA-oligo) or a thiol modified anchor oligonucleotide (Thiol-oligo).

In one aspect, the capture-target hybrid immobilized on the carbon-containing assay surface is coated with a binding reagent that preferentially binds to a binding partner on the assay surface. In one aspect, the capture-target hybrid is immobilized on a streptavidin-coated, carbon-containing assay surface and said surface is coated with a solution containing biotinylated anchor oligonucleotides.

In one aspect, the use of a whole capture-target hybrid that comprises target analytes on its surface limits the off-target recognition of additional proteins that are released into the solution of cells that are lysed. In one aspect, the native conformation of a target analyte on the surface of a whole capture-target hybrid is maintained.

### Direct Coating Method

In one aspect, a direct coating method is provided for preparing a bifunctional assay surface that includes a primary reagent and a secondary reagent. In one aspect, a direct coating method is provided for preparing a bifunctional assay surface that includes a proteinaceous primary reagent and a thiol-containing secondary reagent. In one aspect, a direct coating method is provided for preparing a bifunctional assay surface that includes a capture-target hybrid and a thiol-containing secondary reagent. As used herein, “direct coating” means that either the proteinaceous primary reagent or capture-target hybrid, and thiol-containing secondary reagent are immobilized on the assay surface at the same time. In one aspect, either the proteinaceous primary reagent or capture-target hybrid, and thiol-containing secondary reagent are dispensed onto the assay surface in a coating solution. In one aspect, either the proteinaceous primary reagent or capture-target hybrid, and thiol-containing secondary reagent are dispensed on the assay surface in the same coating solution. In one aspect, the coating solution includes both the proteinaceous primary reagent and the thiol-containing secondary reagent. In one aspect, the coating solution includes both the capture-target hybrid and the thiol-containing secondary reagent. In one aspect, either the proteinaceous primary reagent or capture-target hybrid, and thiol-containing secondary reagent are printed in discrete binding domains on the assay surface. In one aspect, a liquid handling system is used to print the proteinaceous primary reagent or capture-target hybrid and the thiol-containing secondary reagent on the assay surface. In one aspect, the reagents are printed on the assay surface using a non-contact dispenser, including, for example, an ink-jet printer or piezoelectric printer. In one aspect, the reagents are printed on the assay surface using a contact printer, for example, using pins, capillary tubes, or ink stamps.

In one aspect, the primary reagent and the thiol-containing secondary reagent are immobilized on the assay surface through different surface chemistries. In one aspect, the primary reagent is non-covalently immobilized on the assay surface. In one aspect, the primary reagent is non-covalently immobilized on the assay surface by passive adsorption. In one aspect, the primary reagent is covalently immobilized on a carbon-containing assay surface. In one aspect, the primary reagent is immobilized on a carbon-containing assay surface via a binding pair. In one aspect, the primary reagent includes a first member of a binding pair and the assay surface includes a second member of the binding pair. In one aspect, the primary reagent includes biotin and the assay surface is coated with avidin or streptavidin.

In one aspect, the thiol-containing secondary reagent is immobilized on the assay surface by the formation of a bond between the thiol group and a reactive functional group on the assay surface. In one aspect, the thiol-containing secondary reagent is immobilized on a carbon-containing assay surface through a reactive functional group on the assay surface. In one aspect, the thiol-containing secondary reagent is immobilized on the assay surface through a maleimide group. In one aspect, the assay surface is treated to introduce one or more maleimide groups before immobilizing the thiol-containing secondary reagent onto the carbon-containing assay surface.

In one aspect, the primary reagent is printed on the assay surface in an array. In one aspect, a plurality of primary reagents are printed in an array. In one aspect, a plurality of primary reagents are printed in discrete binding domains. In one aspect, a proteinaceous primary reagent or capture-target hybrid is printed on the assay surface in an array. In one aspect, a plurality of proteinaceous primary reagents or capture-target hybrids are printed in an array. In one aspect, a plurality of proteinaceous primary reagents or capture-target hybrids are printed in discrete binding domains.

In one aspect, a plurality of secondary reagents are printed in an array. In one aspect, a plurality of secondary reagents are printed in discrete binding domains. In one aspect, a plurality of thiol-containing secondary reagents are printed in an array. In one aspect, a plurality of thiol-containing secondary reagents are printed in discrete binding domains.

In one aspect, a unique primary reagent and a unique thiol-containing secondary reagent are printed in each binding domain. In one aspect, a unique primary reagent and a common thiol-containing secondary reagent are printed in each binding domain. In one aspect, a plurality of primary reagents are printed in a plurality of primary binding domains. In one aspect, a plurality of thiol-containing secondary reagents are printed in a plurality of secondary binding domains.

In one aspect, the method includes dispensing from about 10 nl, 15 nl, 20 nl, 25 nl, 30 nl, 35 nl, 40 nl, 45 nl, or 50 and up to about 55 nl, 60 nl, 65 nl, 70 nl, 75 nl, 80 nl, 85 nl, 90 nl, 95 nl, or 100 nl of a coating solution on the assay surface. In one aspect, the method includes dispensing from about 25 nl to about 75 nl, about 40 nl to about 60 nl, or about 30 nl to about 50 nl of a coating solution onto the assay surface. In one aspect, the method includes dispensing from about 10 nl, 15 nl, 20 nl, 25 nl, 30 nl, 35 nl, 40 nl, 45 nl, or 50 and up to about 55 nl, 60 nl, 65 nl, 70 nl, 75 nl, 80 nl, 85 nl, 90 nl, 95 nl, or 100 nl of a coating solution on a carbon-containing assay surface. In one aspect, the method includes dispensing from about 25 nl to about 75 nl, about 40 nl to about 60 nl, or about 30 nl to about 50 nl of a coating solution onto a carbon-containing assay surface. In one aspect, the method includes dispensing about 50 nl or 75 nl of a coating solution onto a carbon-containing assay surface.

In one aspect, the coating solution includes from about 50 μg/ml, 75 μg/ml, 100 μg/ml, 125 μg/ml, or 150 μg/ml, and up to about 200 μg/ml, 250 μg/ml, 300 μg/ml, 350 μg/ml, or 400 μg/ml primary reagent, or between about 100 μg/ml to about 400 μg/ml primary reagent. In one aspect, the coating solution includes from about 15 nM, 20 nM, 25 nM, 30 nM, 35 nM, 40 nM, 45 nM, 50 nM, 75 nM, 100 nM, 200 nM, 300 nM, 400 nM, or 500 nM and up to about 500 nM, 1000 nM, 1250 nM, or 1500 nM secondary reagent, or between about 15 nM to about 1500 nM secondary reagent. In one aspect, the coating solution includes from about 100 μg/ml to about 400 μg/ml primary reagent and from about 15 nM to about 1500 nM secondary reagent.

In one aspect, the coating solution includes from about 50 μg/ml, 75 μg/ml, 100 μg/ml, 125 μg/ml, or 150 μg/ml, and up to about 200 μg/ml, 250 μg/ml, 300 μg/ml, 350 μg/ml, or 400 μg/ml proteinaceous primary reagent or capture-target hybrid, or between about 100 μg/ml to 750 μg/ml, about 500 μg/ml to 750 μg/ml, or about 100 μg/ml to about 400 μg/ml proteinaceous primary reagent or capture-target hybrid. In one aspect, the coating solution includes from about 15 nM, 20 nM, 25 nM, 30 nM, 35 nM, 40 nM, 45 nM, 50 nM, 75 nM, 100 nM, 200 nM, 300 nM, 400 nM, or 500 nM and up to about 500 nM, 1000 nM, 1250 nM, or 1500 nM thiol-containing secondary reagent, or between about 15 nM to about 1500 nM thiol-containing secondary reagent. In one aspect, the coating solution includes from about 100 μg/ml to about 400 μg/ml proteinaceous primary reagent or capture-target hybrid and from about 15 nM to about 1500 nM thiol-containing secondary reagent.

In one aspect, the coating solution includes from about 50 μg/ml, 75 μg/ml, 100 μg/ml, 125 μg/ml, or 150 μg/ml, and up to about 200 μg/ml, 250 μg/ml, 300 μg/ml, 350 μg/ml, or 400 μg/ml proteinaceous capture reagent, such as an antibody, an antigen-binding antibody fragment, or a receptor, or between about 100 μg/ml to about 400 μg/ml proteinaceous capture reagent. In one aspect, the coating solution includes from about 15 nM, 20 nM, 25 nM, 30 nM, 35 nM, 40 nM, 45 nM, 50 nM, 75 nM, 100 nM, 200 nM, 300 nM, 400 nM, or 500 nM and up to about 500 nM, 1000 nM, 1250 nM, or 1500 nM thiolated oligonucleotide, or between about 15 nM to about 1500 nM thiolated oligonucleotide. In one aspect, the coating solution includes from about 100 μg/ml to about 400 μg/ml proteinaceous capture reagent and from about 15 nM to about 1500 nM thiolated oligonucleotide.

In one aspect, the coating solution includes a non-ionic detergent such as TRITON X-100. In one aspect, the coating solution includes from about 0.01%, 0.02%, or 0.03% and up to about 0.04% or 0.05% TRITON X-100. In one aspect, the coating solution includes about 0.01%, 0.02%, 0.03%, 0.04%, 0.05% TRITON X-100. In one aspect, the coating solution includes a stabilizing agent such as trehalose. In one aspect, the coating solution includes from about 0.01%, 0.02%, 0.03%, 0.04%, or 0.05% and up to about 0.1%, or 0.5% trehalose. In one aspect, the coating solution includes from about 0.01% to about 0.1%, about 0.01% to about 0.05%, or from about 0.1% to about 0.5% trehalose. In one aspect, the coating solution includes a buffer such as phosphate buffered saline. In one aspect, the coating solution includes a buffer such as Dulbeccos' phosphate buffered saline (DPBS). In one aspect, DPBS includes 2.67 mM KCl, 1.47 mM KH2PO4, 8.1 mM Na2HPO4, and 138 mM NaCl. In one aspect, the buffer includes from about 1 mM, 2 mM, 3 mM, 4 mM or 5 mM and up to about 10 mM, 15 mM or 20 mM ethylenediaminetetraacetic acid (EDTA). In one aspect, the coating solution includes a polyethylene glycol derivative, for example, polyethylene glycol tert-octylphenyl ether which is commercially marketed and referred to herein as TRITON X-100. In one aspect, the coating solution includes from about 100 μg/ml to 750 μg/ml, about 500 μg/ml to 750 μg/ml, or about 100 μg/ml to about 400 μg/ml proteinaceous primary reagent or capture-target hybrid; from about 15 nM to about 1500 nM thiol-containing secondary reagent; from about 0.01% to about 0.1% TRITON X-100; from about 0.1% to about 0.5% trehalose in DPBS; and from about 1 mM EDTA to about 20 mM EDTA. In one aspect, the coating solution includes from about 100 μg/ml to about 400 μg/ml proteinaceous primary reagent or capture-target hybrid; from about 15 nM to about 1500 nM thiol-containing secondary reagent; about 0.03% TRITON X-100 and about 0.4% trehalose in DPBS; and about 10 mM EDTA.

In one aspect, a bifunctional assay surface is prepared by dispensing a coating solution that includes a primary reagent and a thiol-containing secondary reagent onto an assay surface to form a coated assay surface. In one aspect, the assay surface is a carbon-containing assay surface. In one aspect, the coated assay surface is incubated under conditions in which the proteinaceous primary reagent and the thiol-containing secondary reagent are immobilized on the carbon-containing assay surface. In one aspect, the coated assay surface is incubated under conditions in which the capture-target hybrid and the thiol-containing secondary reagent are immobilized on the carbon-containing assay surface. In one aspect, the coated assay surface is incubated overnight. In one aspect, the coated assay surface is incubated at room temperature. In one aspect, the coated assay surface is incubated overnight at room temperature. In embodiments, the coated assay surface is incubated at temperatures above and below room temperature, e.g., between 4° C. and about 37° C. In one aspect, the coated assay surface is incubated for at least about 6, 7 or 8 hours and up to about 10, 11 or 12 hours. In one aspect, the coated assay surface is incubated at a controlled humidity from at least about 30%, 35% or 40% and up to about 40%, 45% or 50%, or from about 30% to about 50%. In one aspect, the coated assay surface is incubated at a controlled humidity of about 30%, 35%, 40%, 45% or 50%. In one aspect, the coated assay surface is incubated at a controlled humidity of about 40%.

### Overcoating Method

In one aspect, an overcoating method is provided for preparing a bifunctional assay surface that includes a primary reagent and a secondary reagent. In one aspect, an overcoating method is provided for preparing a bifunctional assay surface that includes either a proteinaceous primary reagent or capture-target hybrid, and a thiol-containing secondary reagent. As used herein “overcoating” means that the primary reagent and the secondary reagent are immobilized on the assay surface sequentially.

In one aspect, a coating solution that includes the primary reagent is dispensed on the assay surface. In one aspect, the primary reagent is printed in discrete binding domains on the assay surface. In one aspect, the primary reagent is printed on the assay surface in an array. In one aspect, a liquid handling system is used to print the primary reagent on the assay surface. In one aspect, the primary reagent is printed on the assay surface using a non-contact dispenser, including, for example, an ink-jet printer or piezoelectric printer. In one aspect, the primary reagent is printed on the assay surface using a contact printer, for example, using pins, capillary tubes, or ink stamps. In one aspect, an assay surface is obtained on which the primary reagent is already immobilized. In one aspect, the primary reagent is a proteinaceous primary reagent or capture-target hybrid. In one aspect, the primary reagent is a proteinaceous capture reagent.

In one aspect, the assay surface includes a multi-well plate. In one aspect, the assay surface is a carbon-containing assay surface. In one aspect, the assay surface includes a carbon-containing electrode.

In one aspect, the primary reagent is non-covalently immobilized on the assay surface. In one aspect, the primary reagent is non-covalently immobilized on the assay surface by passive adsorption. In one aspect, the primary reagent is covalently immobilized on the carbon-containing assay surface. In one aspect, the capture reagent is immobilized on the carbon-containing assay surface via a binding pair. In one aspect, the primary reagent includes a first member of a binding pair and the assay surface includes a second member of the binding pair. In one aspect, the primary reagent includes biotin and the carbon-containing assay surface is coated with avidin or streptavidin.

In one aspect, the thiol-containing secondary reagent is dispensed onto an assay surface on which a primary reagent has previously been immobilized by dispensing an overcoating solution that includes the thiol-containing secondary reagent on the assay surface. In one aspect, an overcoating solution that includes a thiol-containing secondary reagent is dispensed onto the assay surface to form a coated assay surface. In one aspect, the thiol-containing secondary reagent is immobilized on the assay surface by applying a droplet of the overcoating solution to the assay surface. In one aspect, from about 10 μL, 15 μL, 20 μL, 25 μL, 30 μL, 35 μL, or 40 μL, and up to about 50 μL, 55 μL, 60 μL, 65 μL, 70 μL, 75 μL, 80 μL, 85 μL, 90 μL, 95 μL, or 100 μL of an overcoating solution that includes a thiol-containing secondary reagent is dispensed onto the assay surface. In one aspect, from about 25 μL to about 75 μL of the overcoating solution including the thiol-containing secondary reagent is dispensed onto the assay surface. In one aspect, from about 30 μL to about 50 μL of the overcoating solution including the thiol-containing secondary reagent is dispensed onto the assay surface. In one aspect, the thiol-containing secondary reagent is immobilized on the assay surface by applying a droplet of the overcoating solution to the assay surface and incubating the assay surface while shaking. In one aspect, the coated assay surface is incubated on a shaker at from about 500 rpm, 600 rpm, or 700 rpm and up to about 800 rpm, 900 rpm or 1000 rpm. In one aspect, the coated assay surface is incubated on a shaker at from about 650 rpm, 675 rpm, 700 rpm and up to about 725 rpm, or 750 rpm. In one aspect, the coated assay surface is incubated on a shaker at about 700 rpm, 705 rpm, 710 rpm, 715 rpm, 720 rpm or 725 rpm. In one aspect, the coated assay surface is incubated on a shaker at about 705 rpm.

In one aspect, the overcoating solution includes from about 0.01 μM, 0.05 μM, 0.1 μM, 0.5 μM, 1 μM, or 5 μM and up to about 10 μM, 15 μM, or 20 μM thiol-containing secondary reagent. In one aspect, the overcoating solution includes from about 0.01 μM to about 20 μM thiol-containing secondary reagent. In one aspect, the overcoating solution includes from about 1 μM, to about 10 μM thiol-containing secondary reagent. In one aspect, the overcoating solution includes from about 1 μM, to about 10 μM thiolated oligonucleotide. In one aspect, the overcoating solution includes a buffer selected from: Deprotection-Conjugation Buffer (Meso Scale Diagnostics, LLC), 1×PBS (phosphate buffered saline)/10 mM EDTA (ethylenediaminetetraacetic acid), Diluent 100 (Meso Scale Diagnostics, LLC) or a combination thereof. In one aspect, the deprotection-conjugation buffer includes 10 mM phosphate, pH 7.4, 150 mM NaCl, and 10 mM EDTA.

In one aspect, the thiol-containing secondary reagent is printed in discrete binding domains on an assay surface on which a primary reagent has previously been immobilized. In one aspect, a liquid handling system is used to print the thiol-containing secondary reagent on the assay surface. In one aspect, the thiol-containing secondary reagent is printed on the assay surface using a non-contact dispenser, including, for example, an ink-jet printer or piezoelectric printer. In one aspect, the thiol-containing secondary reagent is printed on the assay surface using a contact printer, for example, using pins, capillary tubes, or ink stamps.

In one aspect, the coated assay surface is incubated under conditions in which the thiol-containing secondary reagent is immobilized on the carbon-containing assay surface through a thiol group. In one aspect, the coated assay surface is incubated from about 1 hour, 2 hours, 3 hours, or 4 hours and up to about 5 hours. In one aspect, the coated assay surface is incubated from about 1 hour to about 5 hours. In one aspect, the coated assay surface is incubated for about 1 hour, 2 hours, 3 hours, 4 hours or 5 hours. In one aspect, the coated assay surface is incubated at room temperature. In one aspect, the coated assay surface is incubated at room temperature for 4 hours.

In one aspect, the thiol-containing secondary reagent is immobilized on the assay surface by the formation of a bond between the thiol group and a reactive functional group on the assay surface. In one aspect, the thiol-containing secondary reagent is immobilized on the assay surface through a maleimide group on the assay surface. In one aspect, the thiol-containing secondary reagent is immobilized on the assay surface by the formation of a covalent bond between the thiol group and a maleimide group on the assay surface. In one aspect, the assay surface is pre-treated to introduce one or more maleimide groups before the thiol-containing secondary reagent is dispensed on the assay surface. In one aspect, a carbon-containing assay surface is pre-treated to introduce one or more maleimide groups before the thiol-containing secondary reagent is dispensed on the carbon-containing assay surface. In one aspect, the assay surface is treated with an amine-to-sulfhydryl crosslinker including SM(PEG)ₙ, wherein n=2 to 24 (ThermoFisher Scientific). In one aspect, n=1, n=2, n=3, n=4 or n=5. In one aspect, n=4.

In one aspect, the thiol-containing secondary reagent includes a thiolated polyethylene glycol (PEG) species, for example, to passivate the assay surface and/or reduce non-specific binding.

In one aspect, the assay surface is pre-treated, for example, with streptavidin, to facilitate immobilization of reagents that include biotin.

### INCORPORATION BY REFERENCE

All references cited herein, including patents, patent applications, papers, textbooks and the like, and the references cited therein, to the extent that they are not already, are hereby incorporated herein by reference in their entirety.

### EXAMPLES

TABLE 1
Anchor oligonucleotides

| | | | | | Additional |
|---|---|---|---|---|---|
| # | Length | Type | Anchor ID | Sequence | modification |
| 1 | 43 | Thiol | Anchor-25+17A-SH | AAG AGA GTA GTA CAG CAG CCG TCA AAA AAA | |
| | | | | AAA AAA AAA AAAA/3ThioMC3-D/ | |
| 2 | 25 | Thiol | Anchor-25-SH | AAG AGA GTA GTA CAG CAG CCG TCA A/ | |
| | | | | 3ThioMC3-D/ | |
| 3 | 25 | Thiol | Anchor-25-peg6- | AAG AGA GTA GTA CAG CAG CCG TCA A/iSp18// | Peg linker |
| | | | SH | 3ThioMC3-D/ | |
| 4 | 25 | Thiol | Anchor-25-2peg6- | AAG AGA GTA GTA CAG CAG CCG TCA A/iSp18// | Peg linker |
| | | | SH | iSp18//3ThioMC3-D/ | |
| 5 | 25 | Thiol | Anchor-25-4peg6- | AAG AGA GTA GTA CAG CAG CCG TCA A// | Peg linker |
| | | | SH | iSp18//iSp18//iSp18//iSp18//3ThioMC3-D/ | |
| 6 | 14 | Thiol | Anchor-14-SH | GTA GTA CAG CAA GA/3ThioMC3-D/ | |
| 7 | 14 | Thiol | Anchor-14-peg6- | GTA GTA CAG CAA GA/iSp18//3ThioMC3-D/ | Peg linker |
| | | | SH | | |
| 8 | 13 | Thiol | Anchor-13-SH | GTA GTA CAG CAA G/3ThioMC3-D/ | |
| 9 | 13 | Thiol | Anchor-13-peg6- | GTA GTA CAG CAA G/iSp18//3ThioMC3-D/ | Peg linker |
| | | | SH | | |
| 10 | 12 | Thiol | Anchor-12-SH | GTA GTA CAG CAA/3ThioMC3-D/ | |
| 11 | 12 | Thiol | Anchor-12-peg6- | GTA GTA CAG CAA/iSp18//3ThioMC3-D/ | Peg linker |
| | | | SH | | |
| 12 | 11 | Thiol | Anchor-11-SH | GTA GTA CAG CA/3ThioMC3-D/ | |
| 13 | 11 | Thiol | Anchor-11-Peg6- | GTA GTA CAG CA/iSp18//3ThioMC3-D/ | Peg linker |
| | | | SH | | |
| 14 | 10 | Thiol | Anchor-10-SH | GTA GTA CAG C/3ThioMC3-D/ | |
| 15 | 10 | Thiol | Anchor-10-Peg6- | GTA GTA CAG C/iSp18//3ThioMC3-D/ | Peg linker |
| | | | SH | | |
| 16 | 9 | Thiol | Anchor-9-SH | GTA GTA CAG/3ThioMC3-D/ | |
| 17 | 9 | Thiol | Anchor-9-Peg6-SH | GTA GTA CAG/iSp18//3ThioMC3-D/ | Peg linker |
| 18 | 9 | Thiol | Anchor-9P-SH | TA GTA CAG C/3ThioMC3-D/ | |
| 19 | 9 | Thiol | Anchor-9P-Peg6- | TA GTA CAG C/iSp18//3ThioMC3-D/ | Peg linker |
| | | | SH | | |
| 20 | 10 | Thiol | Anchor-10-A3-SH | GT/i6diPr/GTA CAG C/3ThioMC3-D/ | 2,6-Diaminopurine |
| 21 | 10 | Thiol | Anchor-10-A3,6- | GT/i6diPr/GT/i6diPr/CAG C/3ThioMC3-D/ | 2,6-Diaminopurine |
| | | | SH | | |
| 22 | 10 | Thiol | Anchor-10-A3,6,8- | GT/i6diPr/GT/i6diPr/C/i6diPr/G C/ | 2,6-Diaminopurine |
| | | | SH | 3ThioMC3-D/ | |
| 23 | 10 | Thiol | Anchor-10-C7-SH | GTA GTA/iMe-dC/AG C/3ThioMC3-D/ | 5-Methyl deoxycytidine |
| 24 | 10 | Thiol | Anchor-10-C7,C10- | GTA GTA/iMe-dC/AG/3Me-dC//3ThioMC3-D/ | 5-Methyl deoxycytidine |
| | | | SH | | |
| 25 | 10 | Thiol | Anchor-10-T2-SH | G/iSuper-dT/A GTA CAG C/3ThioMC3-D/ | Super T |
| 26 | 10 | Thiol | Anchor-10-T2,5-SH | G/iSuper-dT/A G/iSuper-dT/A CAG C/ | Super T |
| | | | | 3ThioMC3-D/ | |
| 27 | 9 | Thiol | A9+1L-34 | T+AGTACAGC/3ThioMC3-D/ | Locked bases |
| 28 | 9 | Thiol | A9+2L-40 | T+AGTA+CAGC/3ThioMC3-D/ | Locked bases |
| 29 | 9 | Thiol | A9+3L-45 | T+AGTA+C+AGC/3ThioMC3-D/ | Locked bases |
| 30 | 9 | Thiol | A9+9L-69 | +T+A+G+T+A+C+A+G+C/3ThioMC3-D/ | Locked bases |
| 31 | 9 | Thiol | A9+8L-61 | T+A+G+T+A+C+A+G+C/3ThioMC3-D/ | Locked bases |
| 32 | 9 | Thiol | A9+7L-59 | T+A+G+T+A+C+A+GC/3ThioMC3-D/ | Locked bases |
| 33 | 9 | Thiol | A9+6L-55 | T+A+G+T+A+C+AGC/3ThioMC3-D/ | Locked bases |
| 34 | 9 | Thiol | A9+4L-48 | T+A+G+TA+CAGC/3ThioMC3-D/ | Locked bases |
| 35 | 9 | Thiol | A9+5L-51 | T+A+G+TA+CA+GC/3ThioMC3-D/ | Locked bases |
| 36 | 9 | Thiol | A9+4L5OM-SH | mU+A+G+TmA+CmAmGmC/3ThioMC3-D/ | 2′-O-Methyl, Locked |
| | | | | | bases |
| 37 | 9 | Thiol | A9+3L6OM-SH | mU+AmGmUmA+C+AmGmC/3ThioMC3-D/ | 2′-O-Methyl, Locked |
| | | | | | bases |
| 38 | 12 | Thiol | Anchor12-OM-SH | mGmTmAmGmTmAmCmAmGmCmAmA/3ThioMC3-D/ | 2′-O-Methyl |
| 39 | 12 | Biotin | Anchor12-Bio-OM | mGmTmAmGmTmAmCmAmGmCmAmA/3Bio/ | 2′-O-Methyl |
| 40 | 25 | Biotin | Anchor-25-Bio | AAG AGA GTA GTA CAG CAG CCG TCA A/3Bio/ | |
| 41 | 9 | Biotin | A9+3L-Bio | T+AGTA+C+AGC/3Bio/ | Locked bases |
| 42 | 9 | Biotin | A9+3L6OM-Bio | mT+AmGmT mA+C+AmGmC/3Bio/ | 2′-O-Methyl, Locked |
| | | | | | bases |
| 43 | 9 | Biotin | A9+4L-Bio | T+A+G+TA+CAGC/3Bio/ | Locked bases |
| 44 | 9 | Biotin | A9+4L5OM-Bio | mU+A+G+TmA+CmAmGmC/3Bio/ | 2′-O-Methyl, Locked |
| | | | | | bases |
| 45 | 9 | PNA | Anchor-9-C11SH | TA GTA CAG C-Lys-C11SH | |
| 46 | 9 | PNA | C11SH-Anchor-9 | C11SH-TAGTACAGC | |

### Example 1. Method for Preparing Bifunctional Assay Surface with a Proteinaceous Capture Antibody and a BSA-Conjugated Anchor Oligonucleotide

This Example describes a method for preparing a bifunctional assay surface on a 96-well 7-spot and 10-spot assay plate (Meso Scale Diagnostics, LLC). Briefly, a proteinaceous capture antibody was printed on the surface and immobilized along with a BSA-conjugated anchor oligonucleotide (BSA-oligo) or a thiol modified anchor oligonucleotide (Thiol-oligo) shown in Table 1.

Briefly, maleimide-modified BSA and thiol-modified anchor oligonucleotide were prepared and conjugated at a molar ratio 1:10. A coating solution was prepared that included from 100 μg/ml to 400 μg/ml capture antibody and from 5 μg/ml to 50 μg/ml of BSA-oligo or Thiol-oligo in a coating solution containing 0.03% TRITON X-100, 0.4% trehalose in phosphate buffered saline (PBS) with 750 μg/ml BSA or without BSA. 50 nl or 75 nl of coating solution was printed on each binding domain of the 10-spot or 7-spot MSD 96-well plate assay surface using a custom dispenser and dried overnight at 40% controlled humidity.

### Example 2. Method for Preparing Bifunctional Assay Surface with a Biotinylated Capture Antibody and Biotinylated Anchor Oligonucleotide

This Example describes an alternate method for preparing a bifunctional assay surface on a streptavidin coated small spot or 10-spot 96-well assay plate (Meso Scale Diagnostics, LLC). In this example, a biotinylated capture antibody was co-immobilized on the streptavidin coated plate (ssSA) with a biotinylated anchor oligonucleotide.

Briefly, the biotinylated capture antibody and biotinylated anchor oligonucleotide were diluted in Diluent 100 to 0.25 μg/ml and 25 pM, respectively, and incubated for 1 hour with shaking at room temperature to be immobilized on SA-coated plate surface.

### Example 3. Overcoating of Plates with Thiol-Modified Anchor Oligonucleotide

This Example describes a method for preparing a bifunctional assay surface by overcoating a MSD V-Plex immunoassay plate (Meso Scale Diagnostics, LLC) on which capture antibodies were immobilized in an array with a overcoating solution that includes a thiol-modified anchor oligonucleotide (Anchor-SH).

Briefly, thiol-modified anchor oligonucleotide (Anchor-SH) with lengths ranging from 9 nucleotides to 43 nucleotides were prepared. Anchor oligonucleotides were deprotected using either dithiothreitol (DTT) or tris carboxy ethyl phosphene (TCEP) reducing reagents according to the product insert instructions (ThermoScientific) to create an anchor-oligo with reactive thiol group. Some of the thiol-modified anchor oligonucleotides included one or more modifications selected from: 2,6-Diaminopurine (2-Amino-dA); 5-Methyl deoxycytidine; Super T (5-hydroxybutynl-2′-deoxyuridine); locked DNA bases; and peptide nucleic acid (PNA) oligonucleotides (See Table 1).

The thiol-modified anchor oligonucleotides were diluted in one of the following buffers: (i) Deprotection-Conjugation Buffer (DCB) (Meso Scale Diagnostics, LLC) containing 10 mM phosphate, pH 7.4, 150 mM NaCl, 10 mM EDTA; (ii) phosphate buffered saline (PBS)/10 mM EDTA; or (iii) Diluent 100

The MSD V-Plex immunoassay plate was washed with phosphate buffered saline (PBS). From 35 μL to 50 μL of the overcoating solution containing 0.5 μM to 20 μM thiol-modified anchor oligonucleotide was added to each well and incubated for 2 hours at room temperature RT with shaking at 705 rpm to immobilize the thiol-modified anchor oligonucleotide in the binding domains on which the capture antibody was immobilized.

### Example 4. Direct Coating of Plates with Capture Antibody and Thiol-Modified Anchor Oligonucleotide

This Example describes a method for preparing a bifunctional assay surface by direct coating of a 10-spot MSD assay plate (Meso Scale Diagnostics, LLC) with a proteinaceous capture antibody and a thiol-modified anchor oligonucleotide (Anchor-SH). In this example, the proteinaceous capture antibody was immobilized by passive adsorption of the antibody to the plate surface and the thiol-modified anchor oligonucleotide was immobilized by covalent bonding of the thiol-group to the plate surface.

Thiol-modified anchor oligonucleotides were prepared as described in Example 3.

A coating solution was prepared that included from 100 μg/ml to 400 μg/ml capture antibody and from 50 nM to 1500 nM thiol-modified anchor oligonucleotide in a coating solution containing 0.03% TRITON X-100, 0.4% trehalose, in phosphate buffered saline (PBS) with 10 mM EDTA. 50 nl of coating solution was printed on each binding domain of the assay surface using a custom dispenser and dried overnight at 40% controlled humidity.

### Example 5. Direct Coating of Plates with Streptavidin and Thiol-Modified Anchor Oligonucleotide

This Example describes a method for preparing a bifunctional assay surface by direct coating of a 10-spot MSD assay plate (Meso Scale Diagnostics, LLC) with a proteinaceous streptavidin and a thiol-modified anchor oligonucleotide (Anchor-SH). In this example, the proteinaceous streptavidin was immobilized by passive adsorption of the streptavidin to the plate surface and the thiol-modified anchor oligonucleotide was immobilized by covalent bonding of the thiol-group the plate surface.

Thiol-modified anchor oligonucleotides were prepared as described in Example 3.

A coating solution was prepared that included 500 μg/ml streptavidin and from 50 nM to 1500 nM thiol-modified anchor oligonucleotide in a coating solution containing 0.03% TRITON X-100, 0.4% trehalose, in phosphate buffered saline (PBS) with 10 mM EDTA. 50 nl of the coating solution was printed on each binding domain of the assay surface using a custom dispenser and dried overnight at 40% controlled humidity.

### Example 6. IL-4 Assay with Overcoated and Direct Coated Anchor Oligonucleotide

Bifunctional assay plates were prepared by the overcoating and direct coating methods described in Examples 3 and 4 with a capture antibody that specifically binds to IL-4 and a thiolated anchor oligonucleotide (Anchor-SH, #2 in Table 1). Assay sensitivity was compared to sensitivity using S-Plex plates (Meso Scale Diagnostics, LLC) without an anchor (No Anchor); streptavidin coated plates (ssSA) with a biotinylated anchor oligonucleotide prepared as described in Example 2; using V-Plex plates (Meso Scale Diagnostics, LLC); and BSA-anchor oligonucleotide prepared as described in Example 1 (data not shown).

Plates with bifunctional surface were washed with PBS or MSD wash buffer (the same wash buffer is used on all wash steps). Dilutions of analyte (calibration curve), including conditions without analyte were prepared and 25 μL to 50 μL was added to the plate and incubated for 1 hour with shaking at 700 RPM at room temperature (RT). The plates were washed, and 40 μL to 50 μL of Turbo Boost Antibody was added to the plate and incubated for 1 hour with shaking at RT. The plates were washed, and 30 μL to 50 μL Enhance solution was added to plate and was incubated for 0.5 hour with shaking at RT. The plates were washed, and 30 μL to 50 μL Detect solution was added to plate and incubated for 1 hour with shaking at 27° C. V-Plex plates were tested according to the MSD product insert.

Assay sensitivity was determined using 4PL fit function; a limit of detection (LOD) was calculated as a concentration correspondent to the signal above the background for 2.5, its standard deviation

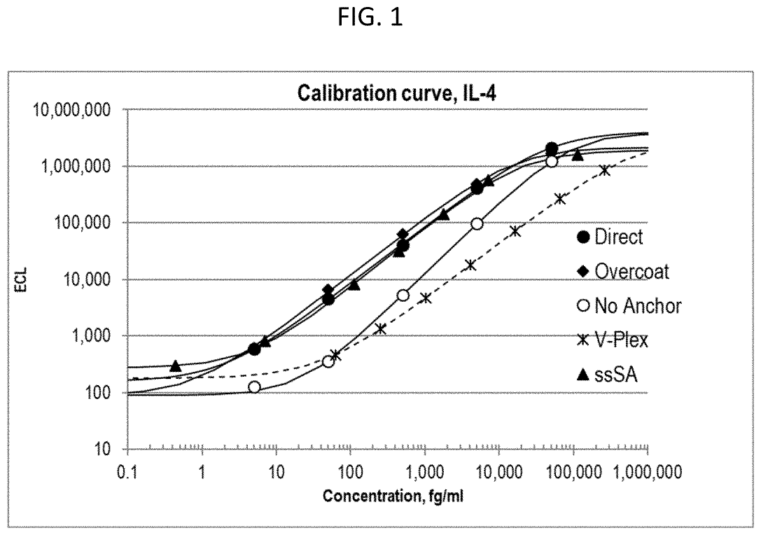

Assay sensitivity was improved about 5-fold for all conditions with an anchor oligonucleotide compared to no anchor control. The results are shown in Table 2 (below) and FIG. 1. Experiments performed using other capture antibodies (IL6, IL10, IL12p70) showed similar assay performance improvements to those seen with the IL4 assay, using overcoating or direct coating approaches of the invention (data not shown).

TABLE 2

| | S-Plex | | | | |
|---|---|---|---|---|---|
| | Direct | Overcoat | No Anchor | ssSA | V-Plex |
| Hill | 1.00 | 1.02 | 1.27 | 1.06 | 1.00 |
| R² | 1.00 | 1.00 | 0.97 | 1.00 | 1.00 |
| LOD (fg/ml) | 1.00 | 0.72 | 16.59 | 1.68 | 19.45 |

### Example 7. IL-4, IL10, and GM-CSF Assay on Streptavidin Plates with Direct Coated Anchor Oligonucleotide and Biotinylated Anchor Oligonucleotide

Bifunctional assay plates were prepared using streptavidin coated plates and the procedure described in Example 2 to immobilize biotinylated capture antibodies specific to IL-4, IL-10, and GM-CSF and biotinylated Anchor. Bifunctional assay plates were also prepared using streptavidin and anchor-SH coated plates described in Example 5 to immobilize biotinylated capture antibodies specific to IL-4, IL-10, and GM-CSF. Assay sensitivity was compared on both types of bifunctional plates, with capture antibodies and anchor attached to the surface via streptavidin-biotin binding (designated in Table 3 as SA), and with capture antibodies attached to the surface via streptavidin-biotin binding, and thiol-modified anchor oligonucleotide immobilized by covalent bonding of the thiol-group the plate surface (designated in Table 3 as SA A-direct).

The assay was performed and assay sensitivity was determined as described in Example 6. Assay sensitivity was similar for both types of bifunctional plates.

TABLE 3

| IL-4 | | | IL-10 | | | GM-CSF | | |
|---|---|---|---|---|---|---|---|---|
| Conc. | SA | | Conc. | SA | | Conc. | SA | |
| fg/ml | A-direct | SA | fg/ml | A-direct | SA | fg/ml | A-direct | SA |
| 20000 | 863129 | 857644 | 35555 | 1136891 | 1034635 | 18600 | 1392142 | 1173792 |
| 2000 | 87298 | 78880 | 3556 | 109257 | 109762 | 1860 | 141568 | 128351 |
| 200 | 9005 | 7934 | 356 | 9039 | 10093 | 186 | 12952 | 11237 |
| 20 | 917 | 686 | 36 | 1114 | 1009 | 19 | 1349 | 1224 |
| 2 | 244 | 235 | 4 | 265 | 226 | 2 | 228 | 198 |
| 0 | 171 | 133 | 0 | 175 | 136 | 0 | 132 | 110 |
| Hill | 1.03 | 1.06 | | 1.04 | 1.05 | | 1.04 | 1.04 |
| R² | 1.00 | 1.00 | | 1.00 | 1.00 | | 1.00 | 1.00 |
| LOD (fg/ml) | 2.10 | 2.41 | | 3.15 | 3.23 | | 1.37 | 1.50 |

### Example 8. Assay Robustness Test

The effect of the thiolated anchor oligonucleotide on S-Plex assay performance was evaluated in an assay robustness test in which a stringent wash was used to assess the ability of the oligonucleotide anchors to hold RCA product on the surface. No sensitivity (Signal/NSB) loss was observed and variability was not increased (% CV) as compared to the S-Plex assay format (data not shown).

### Example 9. Biotin-PEG-SH Reagents

In this example, heterobifunctional thiolated reagents that included biotin and a PEG spacer (Bio-PEG-SH), Nanocs (MW 400, 600 and 1000) were immobilized on the surface of a V-Plex MSD plate and 10-spot 96-well assay plate using overcoating and direct coating methods. For the overcoating method, concentration ranges from 1 μM to 5 μM Bio-PEG-SH were used. For the direct coating method, concentration ranges from 1 μM to 10 μM Bio-PEG-SH were used. The same solvents were used as described in Examples 3 and 4, above. Concentration ranges were based on the thiol-group concentration, measured using Ellmann's reagent (ThermoScientific) according to manufacturer's product insert instructions.

Both the overcoating and direct coating method provided detectable/functional amount of biotin on the plate surface, which was then used to attach biotinylated anchor oligonucleotides (Table 1) bound to streptavidin and make bifunctional S-PLEX plates. Addition of the anchor oligonucleotide via the immobilized biotin allows the anchor sequence to be added at any other step of the S-PLEX protocol assay in addition to the bifunctional surface preparation step described in Examples 1-4 (e.g., together with Turbo Boost Antibodies, Enchance or Detect reagent incubation steps), thereby avoiding potential sample-anchor interference, for example, as anti-DNA antibodies.

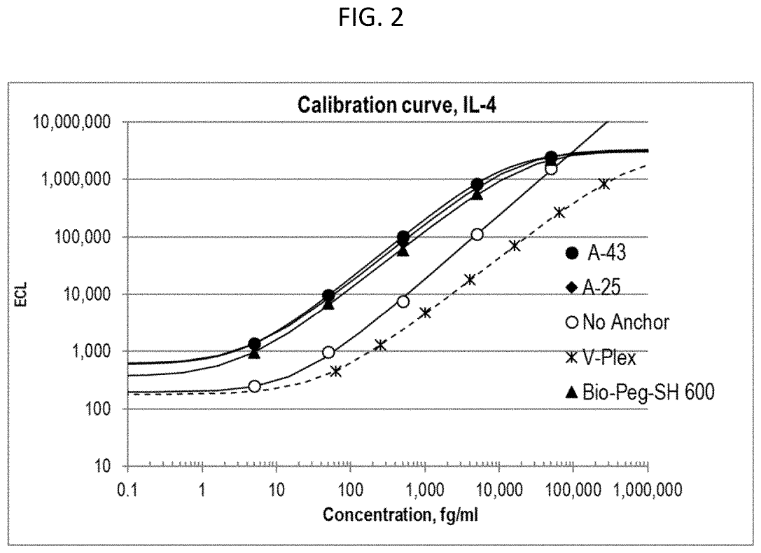

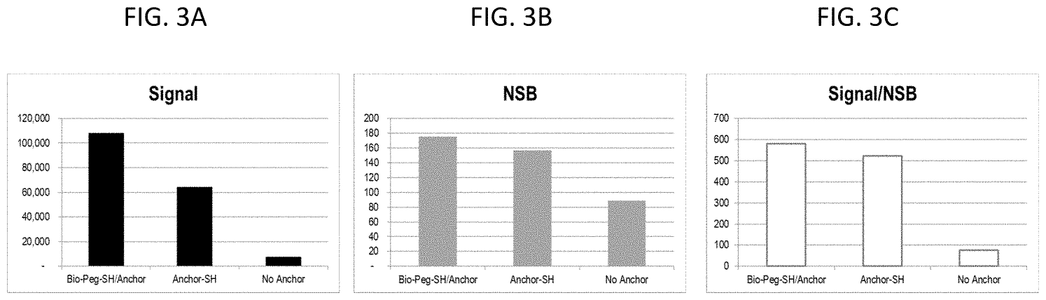

As shown in FIG. 2 and Table 4, plates overcoated with Bio-PEG-SH reagent showed similar performance to those overcoated with Anchor-SH oligo and both performed about 6× better than plates without an anchor oligonucleotide. Plates directly coated with Bio-PEG-SH showed comparable or better performance as plates directly coated with Anchor-SH oligonucleotides when used in an S-Plex assay as described in Example 8 (FIG. 3A, 3B, 3C). Overall sensitivity improvement was about 6-fold compared to No Anchor, similar to Bio-PEG-SH-overcoated plates.

TABLE 4

| | | | | | | | | 2AB | |
|---|---|---|---|---|---|---|---|---|---|
| | | | | | | | | Bio-PEG-SH | |
| 2AB VS plates | | 2AB | | 2AB | | V-Plex | | 600ATS | |
| 5 μM A-43 | | 3.3 μM A-25 | | No Anchor | | QC Data | | | 2AB VS |
| Conc. | 2AB VS | Conc. | 2AB VS | Conc. | 2AB VS | Conc. | | Conc. | BPSH |
| fg/ml | A-43 | fg/ml | A-25 | fg/ml | No A | fg/ml | V-Plex | fg/ml | 600A |
| 50000 | 2479455 | 50000 | 2488953 | 50000 | 1550290 | 260000 | 845130 | 50000 | 2188439 |
| 5000 | 844442 | 5000 | 741328 | 5000 | 110476 | 65000 | 263618 | 5000 | 567452 |
| 500 | 101420 | 500 | 78685 | 500 | 7619 | 16250 | 70663 | 500 | 58717 |
| 50 | 9590 | 50 | 9202 | 50 | 992 | 4063 | 18186 | 50 | 7015 |
| 5 | 1399 | 5 | 1295 | 5 | 254 | 1016 | 4721 | 5 | 940 |
| 0 | 599 | 0 | 623 | 0 | 190 | 254 | 1317 | 0 | 370 |
| | | | | | | 63 | 460 | | |
| | | | | | | 0 | 179 | | |
| Hill | 1.06 | | 1.03 | | 1.12 | | 1.00 | | 1.01 |
| R² | 1.00 | | 1.00 | | 1.00 | | 1.00 | | 1.00 |
| LOD | 1.14 | | 1.29 | | 7.76 | | 19.45 | | 1.04 |
| (fg/ml) | | | | | | | | | |

### Example 10. FITC-PEG-SH Reagents

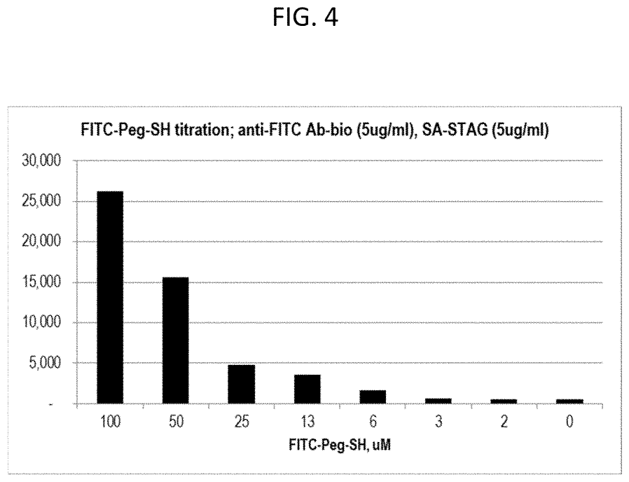

In this example, a fluorescent reagent was immobilized on an assay plate using the overcoating method described herein. Briefly, a fluorescent (FITC-PEG-SH, Nanocs) reagent was immobilized on a V-Plex assay plate surface using the overcoating method described in Example 3. The presence of the fluorescent reagent was detected using a specific anti-FITC antibody. As shown in FIG. 4, signal generation was concentration dependent.

### Example 11. PEG-SH Reagents

In some instances, assay and sample components may stick to surface areas not covered with capture antibodies, increasing background and negatively affecting assay sensitivity. It was hypothesized that surface modification with PEG oligomers could reduce nonspecific binding (NSB) in standard sandwich format assays.

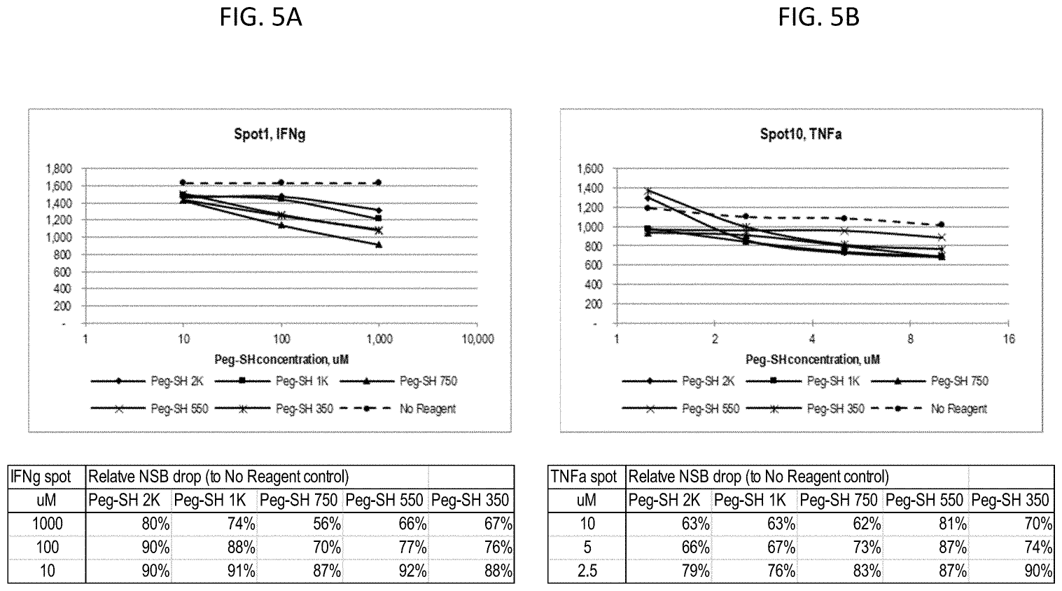

Five PEG-SH reagents, Nanocs (MW 350, 550, 750, 1000 and 2000) were immobilized on an V-Plex assay plate on which capture antibodies were immobilized using the overcoating method described in Example 3 and tested in standard 10-plex sandwich format according to the V-Plex product insert. As shown in FIGS. 5A and 5B, plates overcoated with PEG-SH reduced NSB in a concentration-dependent manner in both IFNg and TNFα assays.

Direct coating of the same reagents on a plate surface with a-IFNg, and a-TNFα antibodies did not generate the same results.

1. A method of preparing a bifunctional assay surface, the method comprising:
a. dispensing a coating solution comprising a proteinaceous primary reagent and a thiol-containing secondary reagent onto a carbon-containing assay surface to form a coated assay surface; and
b. incubating the coated assay surface under conditions in which the proteinaceous primary reagent and thiol-containing secondary reagent are immobilized on the carbon-containing assay surface, wherein the thiol-containing secondary reagent is immobilized on the carbon-containing assay surface through the thiol group of the thiol-containing secondary reagent.

2. The method according to claim 1, comprising dispensing from about 10 nl to about 100 nl, about 25 nl to about 75 nl, about 40 nl to about 60 nl, or about 50 nl of the coating solution onto the carbon-containing assay surface, wherein the coating solution covers the assay surface at a ratio of about 50 nl per binding domain of the assay surface.

3. The method according to claim 1, wherein the coating solution comprises from about 100 μg/ml to 750 μg/ml, about 500 μg/ml to 750 μg/ml, or about 100 μg/ml to about 400 μg/ml proteinaceous primary reagent.

4. The method according to claim 1, wherein the coating solution comprises from about 15 nM to about 1500 nM thiol-containing secondary reagent.

5-8. (canceled)

9. A method of preparing a bifunctional assay surface, the method comprising:
(a) dispensing an overcoating solution comprising a thiol-containing secondary reagent onto a carbon-containing assay surface on which one or more proteinaceous primary reagents are immobilized to form a coated assay surface; and
(b) incubating the coated assay surface under conditions in which the thiol-containing secondary reagent is immobilized on the carbon-containing assay surface through a thiol group.

10. The method according to claim 9, comprising dispensing from about 10 μL to about 100 μL, about 25 μL to about 75 μL, about 30 μL to about 50 μL of the overcoating solution comprising the thiol-containing secondary reagent onto the carbon-containing assay surface.

11. The method according to claim 9, wherein the overcoating solution comprises from about 0.01 μM, 0.05 μM, 0.1 μM, 0.5 μM, 1 μM, or 5 μM and up to about 10 μM, 15 μM, or 20 μM thiol-containing secondary reagent.

12-22. (canceled)

23. The method according to claim 1, wherein the proteinaceous primary reagent is immobilized on the carbon-containing assay surface via a binding pair.

24. The method according to claim 23, wherein the proteinaceous primary reagent comprises a first member of a binding pair and the carbon-containing assay surface comprises a second member of the binding pair.

25-27. (canceled)

28. The method according to claim 1, wherein thiol-containing secondary reagent comprises a thiolated oligonucleotide.

29-37. (canceled)

38. The method according to claim 28, wherein the thiolated oligonucleotide comprises an oligonucleotide sequence having a 5′- and a 3′-end and a thiol group incorporated at the 5′ end (a 5′-terminal thiolated oligonucleotide), at the 3′ end (a 3′-terminal thiolated oligonucleotide), at an internal position of the oligonucleotide, or a combination thereof.

39. The method according to claim 38, wherein the thiolated oligonucleotide comprises deoxyribonucleic acid (DNA), ribonucleic acid (RNA), locked nucleic acid (LNA), peptide nucleic acid (PNA), or a combination thereof.

40. The method according to claim 39, wherein the thiolated oligonucleotide comprises one or more non-natural nucleotide bases.

41. (canceled)

42. The method according to claim 1, wherein the thiol-containing secondary reagent comprises a thiolated member of a binding pair.

43-46. (canceled)

47. The method according to claim 1, wherein the proteinaceous primary reagent comprises a proteinaceous capture reagent that specifically binds to a target analyte.

48-50. (canceled)

51. The method according to claim 47, wherein the proteinaceous primary reagent comprises a first member of a binding pair and the target analyte comprises a second member of the binding pair.

52-54. (canceled)

55. The method according to claim 1, wherein the carbon-containing assay surface comprises a multi-well plate and one or more wells of the multi-well plate comprise one or more electrodes.

56-59. (canceled)

60. A bifunctional assay surface prepared by the method according to claim 1.

61. A method of preparing a bifunctional assay surface, the method comprising:
c. dispensing a coating solution comprising a capture-target hybrid and a thiol-containing secondary reagent onto a carbon-containing assay surface to form a coated assay surface; and
d. incubating the coated assay surface under conditions in which the capture-target hybrid and thiol-containing secondary reagent are immobilized on the carbon-containing assay surface, wherein the thiol-containing secondary reagent is immobilized on the carbon-containing assay surface through a thiol group.

62. A method of preparing a bifunctional assay surface, the method comprising:
e. dispensing an overcoating solution comprising a thiol-containing secondary reagent onto a carbon-containing assay surface on which one or more capture-target hybrids are immobilized to form a coated assay surface; and
f. incubating the coated assay surface under conditions in which the thiol-containing secondary reagent is immobilized on the carbon-containing assay surface through a thiol group.

63-70. (canceled)
